(19) United States
(12) Patent Application Publication
(10) Pub. No.: US 2023/0380414 A1
(43) Pub. Date: Nov. 30, 2023

(54) SYSTEMS, DEVICES AND METHODS FOR DELIVERING AEROSOLIZED FLUOROCARBONS

(71) Applicant: BOUNDLESS SCIENCE, LLC, Lancaster, PA (US)

(72) Inventors: Andrew K. JONES, Lancaster, PA (US); Saibal BANDYOPADHYAY, Lancaster, PA (US); William F. WALKER, Chapel Hill, NC (US)

(21) Appl. No.: 18/031,565

(22) PCT Filed: Oct. 12, 2021
(86) PCT No.: PCT/US21/54545

§ 371 Date: Apr. 12, 2023

Related U.S. Application Data
(60) Provisional application No. 63/090,565, filed on Oct. 12, 2020.

(51) Int. Cl. A01N 1/02

(52) U.S. Cl. CPC A01N 1/0247

(57) ABSTRACT

Disclosed herein are devices, apparatuses, and/or systems for delivering aerosolized fluorocarbons (FCs). The systems can include a container, an aerosolizer, and an introduction assembly. The aerosolizer can be fluidically coupled to the container and configured to aerosolize fluids from the container. The introduction assembly can be fluidically coupled to the aerosolizer and configured to introduce aerosolized fluids into the pulmonary tissue of a subject. Methods include aerosolizing a FC using an aerosolizer, delivering the FC to the pulmonary tissue of the subject, and contacting the pulmonary tissue of the subject with the FC. Some embodiments include cooling the pulmonary tissue. Cooling the pulmonary tissue can provide a therapeutic benefit to a living subject or it can preserve the pulmonary tissue of a deceased subject for transplantation.

### CROSS-REFERENCE TO RELATED APPLICATIONS

This application claims benefit of U.S. Provisional Application No. 63/090,565 filed Oct. 12, 2020, which is hereby incorporated herein by reference in its entirety.

### FIELD

This disclosure relates to pulmonology.

### BACKGROUND

Hypoxetmia, which is typically characterized by a condition where arterial oxygen tension (Pao2) is below normal (normal Pao2=80-100 mmHg), is one of the leading causes of morbidity and mortality. Hypoxemia can be caused by the onset of Acute Respiratory Distress Syndrome (ARDS) or respiratory disease such as asthma, chronic obstructive pulmonary diseases (COPD), pneumonia, or bronchiolitis, or from exposure to chemical irritants/toxins (e.g., smoke inhalation). Asthma attacks and smoke exposure can both cause the inner lining of bronchi and bronchioles swell due to inflammation, thereby causing airways to greatly narrow making it difficult to breath or cough. During pneumonia alveoli fill with fluid due to inflammation caused by bacteria, virus or fungal infection.

Viral pneumonia is often treated with systemic corticosteroids or a composition comprising an antiviral as the active pharmaceutical ingredient (API). Bacterial infections of the pulmonary tissue are generally treated with systemic antibiotics. Inhaled vasodilators are widely used to dilate the airways during an asthma attack. Although systemic treatments can be administered, treatment with inhaled therapeutics are preferred as they can quickly reach alveoli and other lung tissues and have less side effects than systemic therapeutics. Even so, treatment with inhaled therapeutics is complicated by the swelling of lung tissue which inhibits the delivery of inhaled (e.g., volatilized, aerosolized, and/or nebulized) therapeutic compositions. Pneumonia is further complicated by the accumulated fluids in the lung that can resist delivery to and/or absorption by alveolar cells of inhaled therapeutics.

In many instances, patients with hypoxemia are placed on mechanical ventilators to facilitate blood oxygenation and systemic therapeutics are administered intravenously to treat infection. However, ventilator assisted oxygenation of blood requires long term hospitalization and has a substantial (e.g., about 50%) mortality rate. Furthermore, during any global respiratory disease outbreak or pandemic, such as SARS, MERS, or COVID-19, the availability of mechanical ventilator can be insufficient, driving up mortality. Extensive training of healthcare professionals is also required to initiate and maintain ventilator assisted breathing. Hence, there is a need for a minimally invasive approach that can be implemented by patients, home care givers, and/or paramedics for concurrent blood oxygenation and therapeutic delivery to lungs.

Liquid fluorocarbons (FCs) have been known to be useful in delivering oxygen and removing carbon dioxide from the pulmonary tissue since the 1980s, when experiments were conducted on animals, neonates, and adults, FCs are known to be anti-inflammatory and with twice the density of water, can displace water as evidenced by improved oxygenation with aerosolized FC treatment. (Lelmler, 2006).

Liquid ventilation entails filling the lungs with a liquid fluorocarbon and passing oxygen into the liquid while allowing carbon dioxide to exit the liquid. This has typically been done by keeping patients on a ventilator while their lungs were filled or partially filled with fluorocarbons. A liquid ventilation clinical study on patients with Acute Respiratory Distress Syndrome (ARDS) in 2011 ended in failure when mortality results of liquid ventilated patients was slightly worse than patients kept on a simple ventilator. (Kacmarek, 2005).

However, liquid ventilation systems are impractical for human use as (1) they require that patients be sedated to avoid panic and psychological trauma associated with liquid lung filling, (2) they incorporate a prohibitive cost of FCs (˜2/ml) (Sigma-Aldrich), (3) they apply uncomfortable and potentially damaging weight to the chest cavity because of the high lung volume and high density (2 g/ml) of FCs. Hence, there is a need for better treatment options for hypoxemia patients.

### SUMMARY

Disclosed herein are systems, devices, and methods for delivering aerosolized fluorocarbons to pulmonary tissue. Aerosolized fluorocarbon can improve oxygen delivery to a subject without the drawbacks of liquid breathing. FCs are expensive, and the systems and devices disclosed herein are capable of recycling exhaled or expired FCs back the pulmonary tissue to save costs, among other benefits. Furthermore, mixing multiple FCs of varying boiling points enables the user to customize a rapid cooling or warming therapy, which can provide a therapeutic benefit to a living subject. The pulmonary tissue of a deceased subject can be rapidly cooled to better preserve it for transplantation.

Disclosed herein are devices, apparatuses, and/or systems for delivering aerosolized fluorocarbons. The systems include a container, an aerosolizer fluidically coupled to the container and configured to aerosolize fluids from the container, and an introduction assembly fluidically coupled to the aerosolizer and configured to introduce aerosolized fluids into the pulmonary tissue of a subject.

Some embodiments further include a second container and a mixer. The mixer can be fluidically coupled to the first container and to the second container, and the aerosolizer can be fluidically coupled to the mixer, the first container, and the second container. The first container can include a first FC and the second container can include a second FC. In some embodiments, the first EC has a boiling point below 37° C. In some embodiments, the first container and the second container can be housed in one or more disposable cartridges.

In some embodiments, the aerosolizer can aerosolize FCs at a rate of at least 0.5 mL/minute. In some embodiments, the aerosolizer can aerosolize FCs at a rate of at least 2 mL/min. Some embodiments can further include a gas delivery regulator fluidically coupled to the aerosolizer and a gas cannister. The gas delivery regulator can control the flow of a delivery gas to the aerosolizer.

In some embodiments, the introduction assembly comprises a mask. In some embodiments, the introduction assembly comprises a ventilator. In some embodiments, the introduction assembly further comprises at least one one-way valve or flow diverter. For example, a flow diverter can be fluidically coupled to the introduction assembly and the collection system and configured to 1) pass aerosolized fluids from the aerosolizer into the introduction assembly, and 2) pass exhalate or expired fluid from the pulmonary tissue to the collection system.

Some embodiments further include a collection system fluidically coupled to the introduction assembly, the collection system configured to collect and condense exhalate or expired fluid from the pulmonary tissue of the subject into a condensate (for example, via a condenser). In some embodiments, the collection system further comprises a collection tube fluidically coupled to the introduction assembly and the collection system, and a one-way valve or flow diverter fluidically coupled to the introduction assembly but configured to direct exhalate or expired fluid from the pulmonary tissue of the subject into the collection tube. For example, a flow diverter can be fluidically coupled to the introduction assembly and the collection system and configured to 1) pass aerosolized fluids from the aerosolizer into the introduction assembly, and 2) pass exhalate or expired fluid from the pulmonary tissue to the collection system.

In some embodiments, the introduction assembly comprises a ventilator, and the ventilator comprises an exhalation pressure control system. A collection system including a condenser can be fluidically coupled to the ventilator. The condenser can be configured not to interfere with the exhalation pressure control system of the ventilator.

Some embodiments further include a return system fluidically coupled to the collection system, the return system configured to receive condensate from the collection system and return the condensate to the aerosolizer. In some embodiments, the return system includes a separation system in fluid communication with the collection system, the separation system configured to remove water, exhaled gasses, and contaminants from the condensate before delivering the condensate to the return system. In some embodiments, the return system includes a filter for removing water, contaminants, or both from the condensate.

Methods of delivering aerosolized fluorocarbon (FC) to the pulmonary tissue of a subject are also disclosed herein. The methods include aerosolizing a FC using an aerosolizer, delivering the FC to the pulmonary tissue of the subject, and contacting the pulmonary tissue of the subject with the FC. In some embodiments, the FC can be aerosolized at a rate of at least 0.5 mL/minute. In some embodiments, the FC can be aerosolized at a rate of at least 2 mL/minute. Some embodiments can include removing residual FC from the pulmonary tissue. In some embodiments, the FC can be mixed with an active pharmaceutical ingredient before it is delivered to the pulmonary tissue of the subject.

In some embodiments, aerosolizing the can include mixing the FC with a second and aerosolizing a mixture of the first FC and the second FC. The first FC can be selected to have a first boiling point and the second FC can be selected to have a second boiling point. The method can further include balancing the ratio of the first FC to the second FC to engineer a desired boiling point, enthalpy of vaporization, degree of cooling, degree of warming, cooling rate, or waiming rate upon contact of the mixture with the pulmonary tissue. Some embodiments can include mixing the first FC, the second FC, or the mixture of the first and second FC with a delivery gas before the aerosolizing step. Other embodiments can include mixing the delivery gas after the aerosolizing step.

Some method embodiments can include collecting exhaled or expired fluid from the pulmonary tissue of the subject, condensing the exhaled or expired fluid via a condenser, and returning condensed FC to the aerosolizer to be recycled back to the pulmonary tissue of the subject. Some embodiments can include separating the FC from one or more of water, contaminants, and exhaled gas in the exhaled or expired fluid before returning condensed to the aerosolizer. In some embodiments, the step of condensing the exhaled or expired fluid does not change an exhalation pressure control on a ventilator to which the condenser is coupled.

In some method embodiments, the step of contacting the pulmonary tissue further comprises cooling the pulmonary tissue (for example, when at least one FC has a boiling point below 37° C.). The pulmonary tissue can be cooled at a rate of from 0.05° C./minute to 3° C./minute, for example. Cooling the pulmonary tissue can include delivering the FC at a rate of at least 2 mL/minute. The pulmonary tissue of the living subject may be cooled when, for example, the subject is undergoing surgery, has an injury, and/or suffers from ARDS, stroke, heart attack, traumatic brain injury, acute encephalitis, neonatal hypoxia, and/or near drowning, and cooling the pulmonary tissue provides a therapeutic benefit for the subject. The pulmonary tissue can be cooled by, for example, about 2° C. to about 6° C. In some embodiments, cooling the pulmonary tissue reduces inflammation of the pulmonary tissue. In other embodiments, cooling the pulmonary tissue preserving the pulmonary tissue for transplantation. The pulmonary tissue can be cooled by, for example, about 17° C. to about 33° C. to preserve it for transplantation.

Some method embodiments further include performing a lung lavage by delivering mechanical energy to the pulmonary tissue to dislodge one or more of mucus, pus, pollutants, foreign materials, or debris (for example, when the FC has a boiling point above 37° C.). For lung lavage, the volume of aerosolized FC delivered can be, for example, up to 500 mL.

Methods of preserving cadaver pulmonary tissue are disclosed herein. The methods can include selecting a FC with a boiling point below 37° C., aerosolizing the FC using an aerosolizer, delivering the FC to the pulmonary tissue of the subject, and contacting the pulmonary tissue of the subject with the FC, and cooling the pulmonary tissue. To preserve the pulmonary tissue for transplantation, it can be cooled to a range of from 4° C. to 20° C. The FC can be delivered at a rate of at least 2 mL/minute. The pulmonary tissue can be cooled at a rate of from 0.5° C./minute to 3° C./minute.

### BRIEF DESCRIPTION OF THE DRAWINGS

FIG. 1 shows an embodiment of the elements of the disclosed apparatus.

FIG. 2 shows an embodiment wherein a living human subject breathes an aerosolized mixture of two fluorocarbon liquids through a dedicated mask.

FIG. 3 shows an embodiment wherein a living human subject breathes an aerosolized mixture of two fluorocarbon liquids through a dedicated mask. In this embodiment the fluorocarbon mixture is supplemented by oxygen gas.

FIG. 4 shows an embodiment wherein a living human subject breathes an aerosolized mixture of two fluorocarbon liquids through a dedicated mask. In this embodiment the fluorocarbon mixture is supplemented by oxygen gas. Furthermore, the exhaled fluorocarbon gas is condensed into a liquid so that it can be aerosolized and returned to the subject.

FIG. 5 shows one embodiment incorporating a ventilator for use with severely ill or even deceased subjects.

FIG. 6 is a graph showing wave forms associated with the ventilatorshown in FIG. 5.

### DETAILED DESCRIPTION

As used in this application and in the claims, the singular forms “a,” “an,” and “the” include the plural forms unless the context clearly dictates otherwise. Throughout the description and claims of this specification, the word “comprise” and variations of the word, such as “comprising” and “comprises,” means “including but not limited to,” and is not intended to exclude, for example, other additives, components, integers or steps. Additionally, the term “includes” means “comprises.” “Exemplary” means “an example of” and is not intended to convey an indication of a preferred or ideal aspect. “Such as” is not used in a restrictive sense, but for explanatory purposes. “Optional” or “optionally” means that the subsequently described event or circumstance may or may not occur, and that the description includes instances where said event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting aspect the terns are defined to be within 10%. In another non-limiting aspect, the terms are defined to be within 5%. In still another non-limiting aspect, the terms are defined to be within 1%.

Further, the terms “coupled” and “associated” generally means electrically, electromagnetically, fluidically, and/or physically (e.g., mechanically or chemically) coupled or linked and does not exclude the presence of intermediate elements between the coupled or associated items. Such joining may be stationary (e.g., permanent) or moveable (e.g., removable or releasable). The description may use terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

The following description of certain examples of the inventive concepts should not be used to limit the scope of the claims. Other examples, features, aspects, configurations, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain advantages and novel features of the aspects and configurations of this disclosure are described herein. The described methods, systems, and apparatus should not be construed as limiting in any way. Instead, the present disclosure is directed toward all novel and nonohvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed. It will understood that various changes and additional variations may be made and equivalents may be substituted for elements thereof without departing from the scope of the invention or the inventive concept thereof. Certain aspects and features of any given aspect may be translated to other aspects described herein. In addition, many modifications may be made to adapt a particular situation or device to the teachings of the invention without departing from the essential scope thereof. Therefore, it is intended that the invention not be limited to the particular implementations disclosed herein, but that the invention will include all implementations falling within the scope of the appended claims.

Features, integers, characteristics, compounds, chemical moieties, or groups described in conjunction with a particular aspect, configuration, embodiment, or example of the invention are to be understood to be applicable to any other aspect, configuration, embodiment, or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The invention is not restricted to the details of any foregoing aspects. The invention extends to any novel one, or any novel combination, of the features disclosed in this specification (including any accompanying claims, abstract, and drawings), or to any novel one, or any novel combination, of the steps of any method or process so disclosed.

Various publications are referenced in this application. The disclosures of these publications in their entireties are hereby incorporated by reference into this application in order to more fully describe the state of the art to which this disclosure pertains. However, it should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

“Fluorocarbons” are organic compounds containing fluorine and carbon. Fluorocarbons can include other elements besides fluorine and carbon. “Perfluorocarbons” are organic compounds containing only fluorine and carbon. However, the terms are often used interchangeably in the literature. Herein, the term “fluorocarbon” is intended to encompass compounds including fluorine, carbon, as well as other elements. The disclosure is not intended to be limited to solely the use of perfluorinated compounds.

Lung damage and disease are life threatening problems with limited solutions. Lungs are the sole means by which the human body oxygenates blood and removes carbon dioxide. Issues include obstructive diseases (including pneumonia, trauma, cystic fibrosis, pneumothorax), respiratory diseases (including infections, asthma, chronic obstructive pulmonary disease (COPD), acute respiratory distress syndrome (ARDS), tumors (including lung cancer, lymphoma, pleural mesothelioma), complicating conditions which effect the pulmonary tissue (including congestive heart failure, pulmonary edema, and pulmonary hemorrhage), and neonatal diseases (including pulmonary hyperplasia, pulmonary interstitial emphysema, and infant respiratory distress syndrome), and lung poisoning (including corrosive poison gasses).

The inventors have recognized that existing commercial solutions, such as forced air and mechanical ventilators, and proposed solutions, such as liquid ventilation (1) have limitations in “recruiting” or making functional damaged or occluded alveoli to improve gas transfer to and from the blood, (2) have limited ability to remove occluding materials such as alveolar edema, mucus, pus, pollutants, and water from the pulmonary tissue, (3) provide a very limited ability to control the temperature of the pulmonary tissue in living patients, as lowering lung temperature can limit lung and body trauma, and in deceased patients, where cooler pulmonary tissue can be preserved longer for transplant, and (4) provide limited ability to deliver drugs directly to alveoli of patients with compromised pulmonary tissue.

As used herein, the term “subject” encompasses living patients and deceased patients (or cadavers), and is inclusive of any type of animal. The term “fluid” is used to indicate both liquids and gasses.

FCs have a low surface tension, which leads small amounts of FC to actively wet large alveolar surface areas. The inventors disclose a solution wherein a volatile liquid fluorocarbon and fluorocarbon (collectively labeled FC), or a combination thereof, are aerosolized (also referred to atomized or nebulized) and spread throughout the pulmonary tissue using gas combinations which assist in the oxygenation of the pulmonary tissue. The FCs can optionally be mixed or saturated with one or more gasses, such as oxygen and other gasses, to form an appropriate mixture for alveoli. The mixing with gasses can occur either prior to aerosolization or following aerosolization. The aerosolized (atomized or nebulized) FC droplets can be introduced into the pulmonary tissue by an introduction assembly, which can be via (1) spontaneous respiration with a mask (with or without a tracheal tube or endotracheal tube), (2) forced air through a mask (with or without a tracheal tube or endotracheal tube), nose supplied air; or similar system, or (3) a ventilator. The FCs evaporate either quickly or slowly when they strike surfaces on the pulmonary tissue based on their intrinsic boiling points. Optionally, the FCs can be collected from their gaseous state through condensation after their exhalation or forced expiration. Condensed liquids containing FCs can then be filtered to remove water and other contaminants, and optionally mixed or saturated with gas (oxygen and other gases to forrrr an appropriate mixture for alveoli). The condensed and filtered liquids can be returned to be aerosolized and recycled in the pulmonary tissue.

The systems, apparatuses, and methods disclosed herein can quickly cool the pulmonary tissue to provide therapeutic benefits at a rate of up to 1 per minute. Therapeutic cooling is conventionally done with cold systems strapped around body, and can only cool the body at a rate of 1-2° C. per hour. (Varon, 2008). The systems, apparatuses, and methods disclosed herein can also cool the pulmonary tissue of deceased subjects through the same use of a ventilator with a different combination of FCs which vaporize at lower temperatures, cooling at a rate of up to 3° C. per minute. Such a system can quickly cool the pulmonary tissue to close to zero degrees celsius through the use of low-boiling fluorocarbons such as perfiiuorobutane with a boiling point of −1.7° C., though the target temperature for pulmonary tissue planned for transplant is between 10° C. and 20° C. This system can be used to cool and oxygenate the pulmonary tissue of deceased subjects who are placed on a ventilator (or were placed on a ventilator prior to their death). Lung cooling and oxygenation of deceased subjects facilitates lung and other organ preservation for transplant. Conventional techniques for cooling cadaver lungs involves packing ice in the plural space around the lungs and/or flushing the pulmonary vein/arteries with icy salt solution.

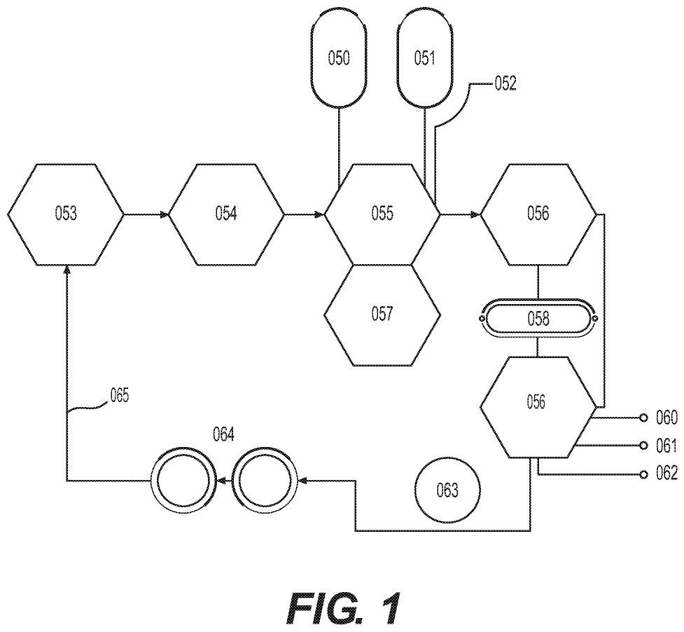

Various embodiments of this disclosure are illustrated in FIGS. 1, 2, 3, 4, and 5. The volume of liquid FCs collected in the pulmonary tissue will be low (typically less than one liter) and most will be volatilized and expired where they will be condensed, filtered, and recycled. Residual FCs in the pulmonary tissue can be removed by (1) inserting a suction tube into the pulmonary tissue, (2) allowing it to slowly vaporize and respirated out, or (3) positioning the subject in a downward direction such that the fluid flows from the pulmonary tissue out of the mouth. Controlled use of specific FCs will allow this system to (a) efficiently oxygenate the blood via improved recruitment (restoring function to damaged or blocked alveoli) of inflamed or occluded alveoli, (b) cool or warm the pulmonary tissue through the enthalpy of vaporization of the specific FCs when they evaporate on contact with lung surfaces, (c) remove mucus and other debris from the pulmonary tissue when sufficient and specific FCs are used to break up these lung contaminants as can be assisted by ultrasound or acoustic energy or mechanical energy, and (d) serve as a vector for therapeutic delivery directly to the alveoli.

In some embodiments, nebulizers can be used to aerosolize FC. Nebulizers are medical devices that generate aerosol from a liquid using compressed gas or piezoelectric energy. Jet nebulizers pull liquid from a liquid reservoir and force the liquid, using compressed gas from a tank or air compressor, through a small restricted opening of a jet nozzle cover which causes nebulization. Ultrasonic nebulizers utilize a piezoelectric motor or piezo-oscillating element. Passing liquid through an aperture mesh or membrane that vibrates at ultrasonic frequencies causes nebulization. Nebulizers typically comprise a housing containing a liquid reservoir and a nebulization chamber with a nebulization generating means, e.g., jet nozzle, vibrating membrane or vibratable mesh, and an aerosol outlet port. Some nebulizers are breath-enhanced and can contain ambient air inlets to more efficiently entrain and remove aerosol. Conventional aerosolizers nebulize around 1 mL/min or less (for aqueous solutions). Aersolizing FC using commercially available nebulizers is a challenging task due to the difference in density and surface tension of FCs as compared with water (for which commercially available nebulizers are designed). For example, using a consumer jet nebulizer, water aerosolizes at 0.2 grams/min (0.2 mL/min), perfluoropentane at 9.2 grams/min (736 mi/min), and perfluorodecalin at 0.88 grams/min (0.5 mL/min). A mix of 50/50 by weight perfluorodecalin/perfluoropentene aerosolized through the same jet nebulizer at 4.2 grams/min (2.0 mL/min).

The disclosure herein encompasses custom aersolizers, inclusive of jet aerosolizers, ultrasonic aerosolizers, forced air aerosolizers, and/or piezoelectric aerosolizers. The aerosolizers herein are able to aerosolize FC (and deliver FC to a subject, introduction assembly, or feed tube), at a rate of at least about 0.5 mL/minute, including at least about 1 mL/minute, at least about 2 mL/minute, at least about 3 mL/minute, at least about 4 mL/minute, at least about 5 mL/minute, at least about 10 mL/minute, at least about 20 mL/minute, at least about 30 mLIminute, at least about 40 mi,/minute, at least about 50 mL/minute, and at least about 60 mL/minute. The aerosolizers herein are able to deliver FC at a rate of up to about 60 mL/minute, including up to about 50 mL/minute, up to about 40 mL/minute, up to about 30 mL/minute, up to about 20 mL/minute, and up to about 10 mL/minute.

Unlike commercially available or known systems, methods and devices, this disclosure encompasses mixing FCs to achieve a desired boiling point, enthalpy of vaporization, degree of cooling, degree of warming, cooling rate, and/or warming rate. Use of one or more volatile FCs with boiling points both below and above body temperature (approximately 37° C.) will allow the introduction of droplets which will ultimately depart the pulmonary tissue as vapor, cooling the pulmonary tissue as they evaporate. Conventional methods of delivering aerosolized FCs have utilized only a single FC and thus, cannot provide a tailored cooling or warming therapy. (Kumar, 2014; Murgia, 2012; Wang, 2014).

Though it may be used, this collection system does not require a collection catheter placed into the lungs. Note that some higher boiling point FCs may collect in the lungs prior to vaporization; but not to a substantial amount. The residual FC in the lungs can be <2000 ml or <1800 ml or <1600 ml or <1400 ml or <1200 ml or <1000 ml or <800 ml or <600 ml or <400 ml or <200 ml or having no residual FC liquid retained in the lungs. Given time, most or all of the FCs in the pulmonary tissue will be exhaled. Any residual FCs in the pulmonary tissue can be removed by inverting the subject and letting it run out of the pulmonary tissue by gravity or by allowing it to slowly vaporize and breathing it out.

Specific FCs or FC combination can be chosen to control the lung temperature. Cooling the body can be an advantageous therapy during surgical procedures and for many injuries, diseases, and disorders, including, but not limited to, ARDS, stroke, heart attack, traumatic brain injury,acute encephalitis, neonatal hypoxia, and near drowning. Cooling the pulmonary tissue can affect an overall cooling of the body. Cooling cadaver pulmonary tissue is advantageous for preserving them for transplantation. Lower boiling point FCs (and combinations thereof) will cool pulmonary tissue as they remove the heat (enthalpy of condensation) as they evaporate. As an example, perfluorobutane, with a boiling point of −1.7 C will cool the body more than perfluoropentane, with a boiling point of 30 C. While their enthalpies of vaporization are similar (perfluorobutane—88KJ/Kg, perfluoropentane—94 KJ/KG), perfluorobutane will continue to evaporate when the lung temperature has dropped below the boiling point of perfluoropenane. As the aerosolized liquids will be chilled, perfluropentane will still cool the pulmonary tissue (Specific Heat—1.05 KJ/Kg*C). Mixtures of these perfluorinated and or fluorinated liquids can be used to optimize the desired boiling point of the liquid to be aerosolized.

The disclosure further encompasses lavage of the pulmonary tissue to clean the pulmonary tissue of debris (such as, but not limited to, pus, mucus, and pollutants). This can be accomplished by filling alveoli with a perfluorinated liquid, and, if desired, adding mechanical energy to the pulmonary tissue to clean out debris to allow proper functioning alveoli. FCs are an especially good liquid for lavage, as they will provide gas transfer to the alveoli while they assist in removing contaminants. Less volatile FCs are typically chosen for this task, as they are desired not to evaporate quickly, but provide a mechanical means of removing mucus, pus, pollutants, foreign materials, and other debris from the pulmonary tissue. Once removed from the alveoli, these materials can either be coughed out or removed with a suction catheter inserted into the pulmonary tissue. Alternatively or additionally, the subject can be positioned so that gravity helps to pull debris down trachea and out the mouth. FCs will help to break up agglomerations so that they can leave the pulmonary tissue. FCs used with lavage in mind will have higher boiling points and be less volatile. Perfluorodecalin (boiling point 140° C.) is an example of a good FC for lavage. The aerosolized droplets of FC will collect in the alveoli to perform their functions, leaving a residual volume of 10 mL to 1000 mL, and will allow for comfortable and effective respiration while assisting in the removal of mucus, particulate, pus, water, and other lung contaminants. Additionally, vibrational energy can be added to the pulmonary tissue once this layer of FC has been applied to the pulmonary tissue to assist in removing the lung contaminants. Such energy can come in the form of mechanical pounding applied to the chest, audible frequencies applied to the pulmonary tissue, or ultrasound energy applied either inside the pulmonary tissue or outside the chest in the vicinity of the lungs. Acoustic energy, either in the form of audible energy or ultrasound, can be applied either from outside the body, to the body wall, or through the air pathway into the pulmonary tissue. The residual FC along with contaminants can be removed from the pulmonary tissue by (1) coughing them out or (2) suctioning them out with a suction catheter or (3) positioning the subject so that gravity assists with removal. Any residual FC can be removed by breathing, allowing the FC to slowly evaporate.

In the present disclosure the fluorinated and/or perfluorinated liquids used can include, but is not limited to, one or more liquids selected from: pertluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1H-PERFLUOROPENTANE, HFA 134a™, HFA227ea™, methyl perfluorobutylether, methyl perfluoropropyl ether (3M Novec 7000™), 2,2,2-trifluoroethanol and combinations thereof. These fluorinated and/or perfluorinated liquids cover boiling point ranges which both above and below the physiological temperature 37° C. In some aspects of the current disclosure, a mixture of at least two fluorinated and/or perfluorinated liquids is used. In some embodiments, at least one component of the fluorinated and/or perfluorinated liquid mixture boils off below body temperature 37° C. and provide cooling effect in the pulmonary tissue. Exhaled vapor help loosening the phlegm from the airways to facilitate the natural breathing. The mixture of fluorinated and/or perfluorinated liquids can have boiling point greater than 20° C., 30° C., 35° C., 40° C., 45° C., 50° C.

Table 1 provides chart of several FCs, detailing their oxygen solubility, specific heats for both liquid and gas, boiling points, and enthalpy of vaporization. Table 2 provides the boiling point of some additional FCs.

TABLE 1
Properties of certain FCs

| | | | | | | Spec. | Spec | | |
|---|---|---|---|---|---|---|---|---|---|
| | | | | | O2 | Ht Liq | Ht gas | Boiling | Enthalpy |
| | | At | Den | Viscosity | Solubility | C(KJ/ | C(KJ/ | point ° C. | of Vap. |
| | liquid | Wt | (g/ml) | cPs | ml/100 ml | Kg/C) | Kg/C) | (C) | H(Kj/Kg) |
| 1 | C4F10 | 238 | 1.63 | 0.012 | | 0.53 | 0.69 | −1.8 | 88 |
| 2 | CSF12 | 288 | 1.59 | 0.460 | 54 | 1.05 | 1.02 | 30 | 94 |
| 3 | C6F14 | 338 | 1.69 | 0.979 | | 0.71 | 0.78 | 69 | 93 |
| 4 | C7F16 | 388 | 1.75 | 0.900 | | 0.64 | 0.82 | 82 | 90 |
| 5 | C8F18 | 438 | 1.77 | 1.800 | | | | 377 | 76 |
| 6 | C10F18 | 486 | 1.92 | 5.100 | 49 | 1.05 | | 142 | 85 |

TABLE 2
Boiling points of additional FCs

| Perfluorinated | Boiling point |
|---|---|
| liquids | ° C. |
| perfluoropropane | −36.7 |
| Perfluorobutane | −1.7 |
| perfluoropentane | 28.0 |
| Perfluorohexane | 56.0 |
| perfluoroheptane | 56.0 |
| Perfluorooctane | 103.0 |
| Perfluorodecalin | 142 |
| Perfluoroperhydrophenanthrene | 215 |
| Perfluorooctylbromide | 142 |
| perfluoro tributyl amine | 178 |
| perfluorotripentyl amine | 215 |

The disclosure further encompasses mixing therapeutics with the FCs. Compositions can include of active pharmaceutical ingredient (API) mixed with the FCs prior to aerosolization (nebulization) and delivered to alveoli through the FC. The API composition can be miscible with FC combination. This can occur naturally, involve chemical modification with hydrophobic chains, or creation of a reverse emulsions using a surfactant or dissolving API in FC using a cosolvent which can be mixed with FCs.

The disclosure further encompasses recycling FCs via condensation and filtration. FCs are expensive. Commercially available or otherwise known systems, apparatuses, and/or methods do not envision recycling expired or exhaled FCs. For example, this can be a closed loop system.

Various embodiments of this disclosure are illustrated in FIGS. 1 through 5. All of the embodiments disclosed herein can include any number of pumps, valves, inlets, outlets, and power sources for facilitating the flow of the fluids through the system. The drawings are merely exemplary and certain features may be used singularly or in combination with other features. The drawings are not necessarily drawn to scale.

FIG. 1 shows a schematic for a closed loop embodiment that is configured to deliver FCs to a subject, collect them from exhalate or expired fluids, recycle, sterilize, and return them to the subject. First container 50 holds a first FC, and second container 51 holds a second FC. The first and second containers are configured to send the first and second FCs to a mixer 55. The mixer 55 can take the form of a reservoir, in some embodiments. In some embodiments, the overall system can be set up to receive instructions from a user and send specified volumes of each of the first and second FCs to mixer 55 (to achieve a custom boiling point or enthalpy of vaporization from the FC mixture and thus provide tailored cooling or lavage therapy to the subject). Optionally, the mixer 55 can also be coupled to a line 52 for incorporating an active pharmaceutical ingredient (API). The mixer 55 can also be attached to a chiller 57.

Mixer 55 is fluidically coupled and configured to delivery the FC mixture to aerosolizer 58. The aerosolizer 58 is designed especially for the aerosolization of FC mixtures, and can aerosolize FCs at a rate of at least 10 mL/minute. The aerosolizer 58 can include a jet nebulizer, an ultrasonic nebulizer, piezoelectric, or forced air components.

A gas delivery regulator 56 can be coupled to the system either before or after the FC mixture is delivered to the aerosolizer 58. The gas delivery regulator 56 can control the flow of a delivery gas, such as oxygen, from an attached gas cannister. In the depicted embodiment, the gas delivery regulator 56 is coupled to alterative introduction assemblies 60, 61, or 62 for introducing the aerosolized. FC mixture into pulmonary tissue 63 of the subject. The introduction assembly can include a ventilator 60, or a mask for spontaneous breathing 61, or a forced air mask 62, any of which are capable of introducing the IT mixture into the pulmonary tissue 63 of the subject.

Next, a collection system collects and condenses the exhal ate or expired fluid from pulmonary tissue 63 of the patient. The collection system can include a one-way valve 64 that transfers exhaled or expired fluid to a condenser 53 via collection tube 65. The condenser 53 collects and condenses exhaled or expired gasses into liquids. The collection system 53 then sends the condensate, which includes the exhaled FC mixture back to mixer 55 and aerosolizer 58 via a return system 54. Some embodiments of return system 54 can include a separation system that removes water, exhaled gasses, and contaminants from the condensate before sending it back to mixer 55. Some embodiments of return system 54 can include a filter that removes contaminants and water from the condensate before sending it back to mixer 55.

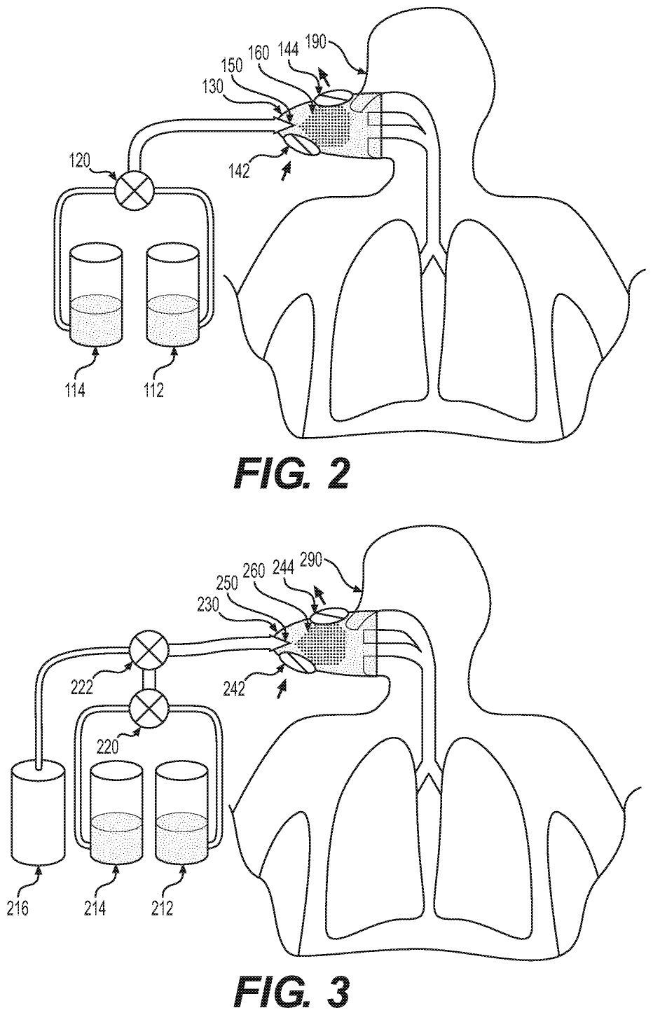

FIG. 2 depicts an embodiment of the apparatus in use by a subject 190. FIG. 2 shows a first container 112 containing a first liquid FC and a second container 114 containing a second liquid FC. The first and second containers 112, 114 are fluidically coupled to a mixer 120 (such as a reservoir). The relative amounts of the FCs can be adjusted to achieve a desired evaporation temperature of the mixture for tailored cooling, heating, or lavage of the pulmonary tissue. FIG. 2 depicts an introduction assembly including a mask 130 that covers the subject's mouth and nose for spontaneous breathing. The mask 130 includes two one-way valves. The first one-way valve 142 allows air to enter the mask 130 when the subject 190 inhales. The second one-way valve 144 is configured to allow air to leave the mask 130 when the subject 190 exhales. The mask 130 is coupled to an aerosolizer 150, which is configured to create a FC aerosol 160 upon entry into mask 130. The FC aerosol 160 enters subject's nose and mouth. The embodiment depicted in FIG. 2 does not incorporate a collection system or a return system. Instead, the subject 190 exhales the FCs back into the environment via the second one-way valve 144. In another embodiment, the mask 130 can be replaced and/or supplemented by a tracheal tube with associated external components.

FIG. 3 depicts another embodiment of the apparatus in use by a subject. FIG. 3 includes a first container 212 containing a first FC liquid and a second container 214 containing a second liquid FC. The first and second containers 212, 214 are fluidically coupled to a mixer 220 (such as a reservoir). The relative amounts of the FCs can be adjusted to achieve a desired evaporation temperature of the mixture for tailored cooling, heating, or lavage of the pulmonary tissue. A delivery gas from cannister 216 can be introduced to the system at a second mixer/gas delivery regulator 222, which can control the amount of gas (oxygen gas, for example, or air with a higher than normal amount of oxygen) that enters the FC mixture on its way to aerosolizer 250. The aerosolizer 250 aerosolizes the FC mixture as it enters mask 230. The mask 230 can include two one-way valves. The first one-way valve 242 allows air to enter the mask 230 when the subject 290 inhales. The second one-way valve 244 is configured to allow air to leave the mask 230 when the subject 290 exhales. The FC aerosol 260 enters subject's nose and mouth. The embodiment depicted in FIG. 3 does not incorporate a collection system or a return system. Instead, the subject 290 exhales the FCs back into the environment via the second one-way valve 244. In one embodiment, the gas delivery regulator 222 can be used to alternate between the application of the fluorocarbon mixture (liquids to be aerosolized) and the oxygen or oxygen enriched gas. In one embodiment, the mask 230 can be replaced and/or supplemented by a tracheal tube with associated external components. In one embodiment, the delivery gas from cannister 216 can be replaced and/or supplemented by an oxygen concentrator.

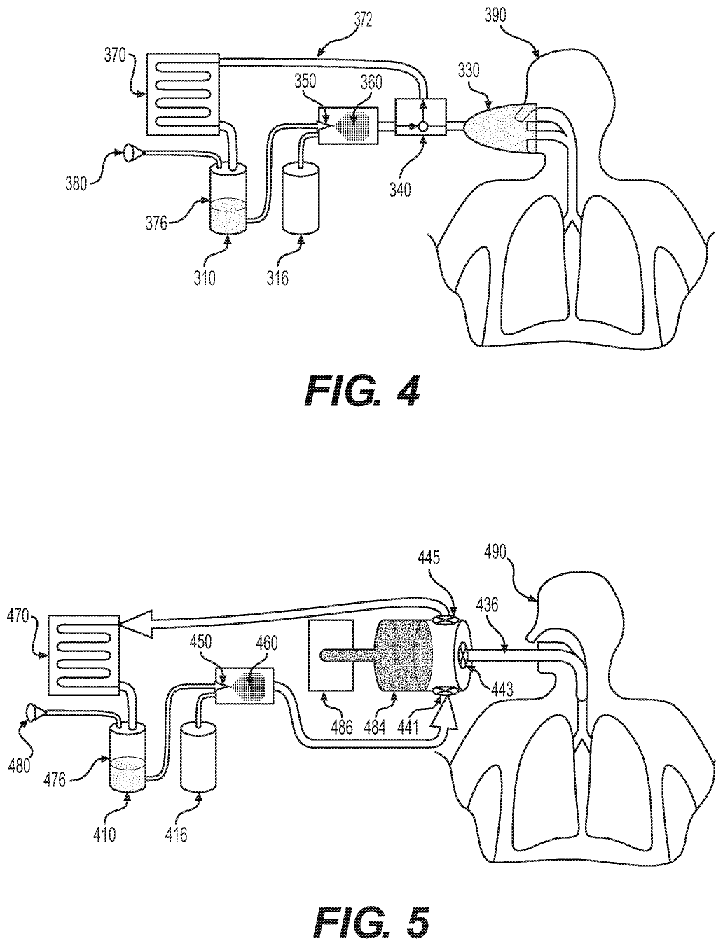

Another embodiment of the apparatus is depicted in FIG. 4. Here, the apparatus only includes a single container 310 for FCs. The PCFs contained in the single container 310 can be a single FC, or a mixture of two or more FCs. In contrast to the system shown in FIG. 3, here, delivery gas (such as oxygen or air with a higher than normal amount of oxygen), from gas cannister 316 is mixed into the FCs after they have been aerosolized at aerosolizer 350. Thus, FC/oxygen gas mixture 360 contains both aerosolized FCs and oxygen gas. The aerosolizer 350 is coupled to a flow diverter 340 that allows the mixture 360 to be inhaled into mask 330 and that diverts exhalate into a col lection system. The collection system includes a collection tube 372 that leads the exhalate to a condenser 370, which is designed to condense gas FCs into its liquid phase and return them to container 310. In some embodiments, container 310 is part of a return system, and can also include a separation system 376 that separates the liquid perfluorcarbon mixture from other exhaled gasses and liquids. The separation system can include a filter for contaminants and exhaust 380 for exhaled gasses. Or the filter can be in line with the tube that sends the separated FCs back to the aerosolizer 350. In another embodiment, the mask 330 can be replaced and/or supplemented by a tracheal tube. In another embodiment, element 316 can be replaced and/or supplemented by an oxygen concentrator.

The embodiment depicted in FIG. 5 is configured for use with a ventilator 486. Here, the apparatus only includes a single container 410 for FCs. The PCFs contained in the single container 410 can be a single or a mixture of two or more FCs. Delivery gas (such as oxygen, or air with a higher than normal amount of oxygen), from gas cannister 416 is mixed into the FCs after they have been aerosolized at aerosolizer 450. Thus, FC/oxygen gas mixture 460 contains both aerosolized FCs and oxygen gas. Mixture 460 is delivered to a controllable valve 441. A first controllable valve 441 is coupled to the ventilator 486 and configured to allow the gas/aerosolized fluorocarbon mixture to enter the piston 484 of ventilator 486 in order to be pushed into the subject's airway via an endotracheal tube 436 employed to connect to the airway of subject 490. A second controllable valve 443 is coupled to the ventilator 486 and positioned between the piston 484 of ventilator 486 and the endotracheal tube 436 to either connect or block the connection. A third controllable valve 445 is coupled to the ventilator 486 and configured to allow exhalate to pass from the ventilator piston 484 through a collection tube 472 and to the condenser 470. The condenser 470 is designed to condense gas FCs into its liquid phase and return them to container 410. In some embodiments, container 410 is part of a return system, and can also include a separation system 476 that separates the liquid perfluorcarbon mixture from other exhaled gasses and liquids. The separation system can include a filter for contaminants and an exhaust 480 for exhaled gasses. Or the filter can in line with the tube that sends the separated FCs back to the aerosolizer 450. In another embodiment, gas cannister 416 can be replaced and/or supplemented by an oxygen concentrator.

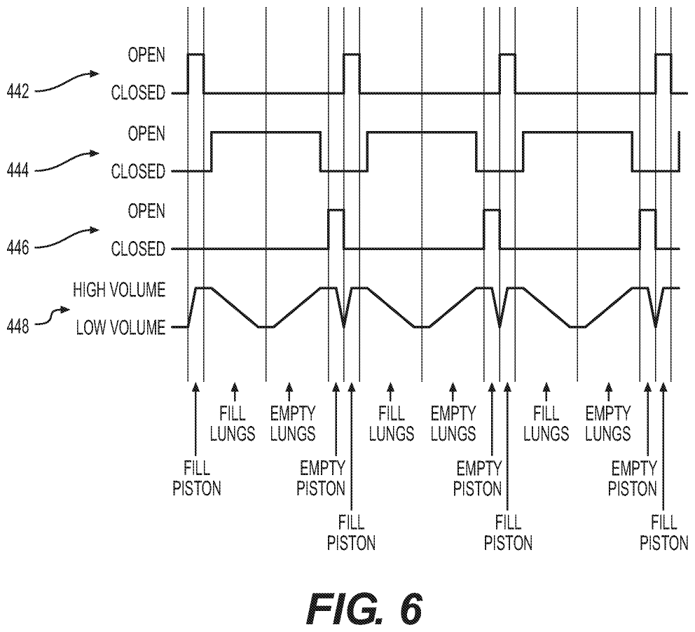

FIG. 6 is a graph showing wave fot ins associated with the ventilator 486 of FIG. 5. Wave form 485 is associated with the cycling of air from the ventilator 486 and into the pulmonary tissue of subject 490. Wave form 442 is the wave form of the first controllable valve 441 allowing aerosolized fluorocarbon liquid into ventilator airstream. Wave form 444 is the wave form of the second controllable valve 443 allowing aerosolized fluorocarbon liquid to flow to and from the ventilator. Wave form 446 is the wave form of third controllable valve 445 taking exhaled fluorocarbon into the condenser 470.

### Aerosolization of the Liquid

Aerosol drop diameter can be between 1-5 um, 5-10 um, 10-15 um, 15 um-100 um, or 100 um-1 mm. The following niechanisms of delivery are brought into consideration when determining desired aerosol drop diameter: inertial impaction, gravitational sedimentation (settling) and diffusion. For FCs, drop diameter can optionally be closer to 1 micron.

Water can be aerosolized or vaporized either separately or together with the other liquids to be aerosolized to provide moisture to the pulmonary tissue.

### Aerosolization

Aersolization of the liquids to be aerosolized can be accomplished in several ways:
(a) Ultrasonic energy is applied to the liquids to be aerosolized (including piezoelectric energy),
(b) High pressure air is applied around the liquid create an air stream which draws in droplets

The liquids to be aerosolized can be aerosolized in droplets with diameters less than 1 micrometer, into larger droplets, between 1 and 5 micrometers, or in larger droplets between 5 and 25 micrometers in diameter.

### Closed Loop

One embodiment is configured as a closed loop with regard to the FCs. Liquids in one or more reservoirs are aerosolized and passed into the pulmonary tissue via an introduction assembly, such as a mechanical ventilator or mask (for either spontaneous or forced breathing), possibly also through a tracheal tube or endotracheal tube during the inhalation phase of respiration. Gases required by the body such as oxygen are combined with the liquid either before or after aerosolization. The liquids evaporate in the pulmonary tissue and are returned to the circuit during subject exhalation phase of respiration. The gaseous liquids are condensed, subsequent liquids filtered of water and other contaminants, and returned to the reservoir where they are mixed and aerosolized for inhalation back into the pulmonary tissue. If used in conjunction with a ventilator, the condenser can be used either before or after the pressure control valve on the exhalation branch of the ventilator. The condenser is designed not to interfere with the ventilator's exhalation pressure control. The system will supply aerosolized liquid saturated with specific gases during inhalation and receive exhaled gases during exhalation. The continuous loop is illustrated in FIG. 1. Various potential designs for this system are included in FIGS. 2, 3, 4, and 5.

To facilitate this continuous cycle, the apparatus can include a valve thatdelivers and receives the aerosolized liquid by:
(a) pushing the aerosolized mixture into the mask or ventilator during subject inhalation, and
(b) drawing the exhaled vapors and gases from the mask or ventilator during subject exhalation.

The design of a condenser that is both efficient (captures a high proportion of vapor) and presents a low gas flow resistance (to avoid interference with the ventilator and subject respiration) is a substantial engineering challenge. In one approach, an array of small condensers can be placed in parallel. The net gas flow resistance of such a system can be quite low because placing many gas channels in parallel will reduce the total resistance. Since each small condenser presents a large surface area relative to the gas volume flowing through it, the capture efficiency is high.

### Mixing Liquids to Change Boiling Point

The boiling point of the liquid can be engineered by mixing two or more unique miscible liquids (such as FCs) to arrive at a boiling point appropriate for lung cooling. By combining liquids, a boiling point can be arrived at which optimizes the desired lung cooling (or lack thereof) and preventing lung damage from cooling while still providing a gaseous return of the liquids to this apparatus. As an example, mixing liquid perfluoropentane with perfluorodecalin would provide a mixture with a boiling point above approximately 28 C but below approximately 140 C, depending on the combination of the two liquids.

### Different Liquids Over Time

This apparatus could aerosolize different liquids at different times for the same subject. As an example, the system could aerosolize perfluorodecalin (boiling point˜140 C) into the pulmonary tissue for 20 minutes to provide a less volatile layer of perfluorocabons throughout the pulmonary tissue to recruit alveoli (make more alveoli available for gas transfer to blood in sick subject). Following this layering of a higher-boiling fluorocarbon, a more volatile FC such as perfluoropentane (boiling point˜30 C) can be aerosolized into the pulmonary tissue. The lower boiling point perflourocarbons would evaporate at a much faster rate, returning to the apparatus for condensation and reuse in the subject. The higher boiling point fluorocarbon, such as perfluorodecalin, would also evaporate out of the pulmonary tissue, albeit at a much slower rate over hours or perhaps days. This residual fluorocarbon would not diminish the functionality of the pulmonary tissue.

### Removing Residual Liquids

In some embodiments of this disclosure, the liquid for aerosolization can contain liquids higher than body temperature (approximately 37 C) and can accumulate in the pulmonary tissue. Such liquids can be useful for several reasons:
(a) provide liquid through which mechanical energy can be applied to remove alveolar edema, mucus, phlegm, pus, and other contaminants from the alveoli
(b) provide a layer of fluorinated or perfluorinated liquid on the pulmonary tissue to improve blood oxygenation
(c) allow for the heating of pulmonary tissue through the use of warm liquid to be aerosolized
   Residual liquids can be removed from the pulmonary tissue in one of three ways:
(a) inserting a suction line into the pulmonary tissue and suctioning the liquid out
(b) inverting the subject such that his lungs are above his mouth and draining the liquid out of his mouth using gravity
(c) allowing the liquid to slowly evaporate from the pulmonary tissue

### Recycling for Future Subjects

The apparatus can have a recycling system, or a return system, to isolate the individual component liquids in the liquid for aerosolization after use in a subject. This apparatus can be a part of or a separate apparatus to the apparatus used on the subject. This system will separate and sterilize the liquids for use on future subjects, allowing for the recycling of these liquids in multiple subjects.

### Cartridges for Individual Subjects

The apparatus can have a disposable cartridge which contains liquids for a subject and flow paths of the liquid through the machine which could be contaminated through subject use. By replacing such a cartridge, a completely sterile system could be quickly established for each new subject. In some embodiments, each of the FCs intended to be mixed are housed in a separate disposable cartridge. In some embodiments, FCs are premixed and contained in a single disposable cartridge.

### Cooling the Pulmonary Tissue

To cool the body as a therapy, the pulmonary tissue can be cooled by about 2° C. to about 6° C., (including about 2° C., about 2.5° C., about 3° C., about 3.5° C., about 4° C., about 4.5° C., about 5° C., about 5.5° C., and about 6° C.) to reach a temperature ranging from about 31° C. to about 35° C.

To cool cadaver pulmonary tissue to preserve them for later transplantation, the pulmonary tissue can be cooled by about 17° C. to about 33° C., (including about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., and about 33° C.) to reach a temperature of about 4° C. to about 20° C.

Advantageously, the methods disclosed herein enable a cooling rate of from about 0.05° C. per minute to about 3° C. per minute (including about 0.05° C. per minute, about 0.25° C. per minute, about 0.5° C. per minute, about 0.75° C. per minute, about 1.0° C. per minute, about 1.25 ° C. per minute, about 1.5° C. per minute, about 1.75° C. per minute, about 2.0° C. per minute, about 2.25° C. per minute, about 2.5° C. per minute, about 2.75° C. per minute, and about 3.0° C. per minute).

### Portable System

The systems on the apparatus can be constructed in a small manner in which such a system is completely ambulatory and could be used in an ambulance or carried with a subject as he/she travels. An example of such an ambulatory system could operate in a device the size of a student's backpack. Such a system might include a battery pack to enable use when transferring the subject between locations with reliable power.

### Mask

The mask used for the aerosolizer will minimize the release of aerosolized droplets from either the medication of the subject into the environment. The method to remove such exhaled droplets will be to capture them in a finely woven cloth, paper, or polymer material, either woven or nonwoven, which is used as a face mask for the subject. The mask can do this by accomplishing the following: (1) the mask can be make a flush fitting around a subject's mouth and nose. Optimizing flush fit over the user's nose either with (a) a wire form which can be adjusted to a user's nose, (b) a contoured tailoring of the mask such that it fits snugly over a subject's nose, and/or (c) any elastomeric material in the nose region which draws the mask close to the subject's nose; (2) the mask can be constructed of a hydrophobic or hydrophilic material with a mesh small enough to capture aerosolized droplets prior to leaving the mask. For example, the material can be silk, chiffon, cotton, a cotton synthetic mix, a synthetic material such as polyester, polytetrafluoroethylene, nylon, and polyvinylchlodde, among others. As aerosolizer or nebulizer droplets typically range in size from 2 micrometers to 5 micrometers, the mean size of the material openings can be 2 micrometers in diameter or less. This pore size can also be accomplished by using multiple layers of fabrics of larger pore size; (3) the mask can have a one-way valve attached to its surface to allow additional air to enter the mask from the outside while preventing aerosolized droplets from leaving the mask through the valve; (4) the mask can have an attachment to connect the aerosolized medicine output of the nebulizer to the inside of the mask without leaking aerosolized material to the outside environment. This attachment can take many forms, one of which is a male hub around which the exhaust tube of the nebulizer is firmly slid.

Partial list of Fluorinated and Perfluorinated liquids which can be used individually or in combination with one or more liquids for use in this disclosure.

Fluorinated diethylene glycol monomethyl ether, 98%
(1H,1H-Perfluoro-3,6-dioxaheptan-1-ol)

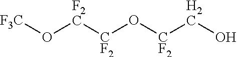 [SMILES: OCC(F)(F)OC(F)(F)C(F)(F)OC(F)(F)F]

| | | Molecular | | |
|---|---|---|---|---|
| Molecular Formula | CAS# | Weight | Physical State | Boiling Point |
| C5H3F9O3 | 330562-43-1 | 282 | Clear liquid | 117° C. |

1H, 1H-Perfluoro-1-pentanol, 98%
(1H, 1H-Nonafluoro-1-pentanol)

| Molecular Formula | CAS# | Molecular Weight | Physical State | Boiling Point |
|---|---|---|---|---|
| C5H3F9O | 355-28-2 | 250 | Clear liquid | 110-111° C. |

1H,1H-Perfluoro-1-hexanol, 98%
(1H,1H-Perfluoro-1-hexyl alcohol)

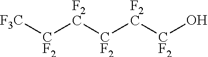 [SMILES: OC(F)(F)C(F)(F)C(F)(F)C(F)(F)C(F)(F)C(F)(F)F]

| Molecular Formula | CAS# | Molecular Weight | Physical State | Boiling Point |
|---|---|---|---|---|
| C6H3F11O | 423-46-1 | 300 | Clear liquid | 130-131° C. |

Fluorinated triethylene glycol monomethyl ether, 98%
(1H,1H-Perfluoro-3,6,9-trioxadecan-1-ol)

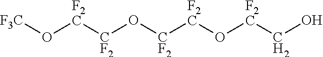 [SMILES: OCC(F)(F)OC(F)(F)C(F)(F)OC(F)(F)C(F)(F)OC(F)(F)F]

| Molecular | | Molecular | Physical | Boiling |
|---|---|---|---|---|
| Formula | CAS# | Weight | State | Point |
| C7H3F13O4 | 147492-57-7 | 398 | Clear liquid | 140-142° C. |

Fluorinated diethylene glycol monobutyl ether, 98%
(1H,1H-Perfluoro-3,6-dioxadecan-1-ol)

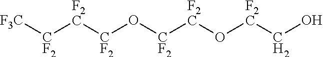 [SMILES: OCC(F)(F)OC(F)(F)C(F)(F)OC(F)(F)C(F)(F)C(F)(F)C(F)(F)F]

| Molecular | | Molecular | Physical | Boiling |
|---|---|---|---|---|
| Formula | CAS# | Weight | State | Point |
| C8H3F15O3 | 152914-73-3 | 432 | Clear liquid | 150° C. |

Perfluoro-3,7-dimethyloctan-1-ol, 97%
(3,7-Bis(trifluoromethyl)tridecafluorooctanol)

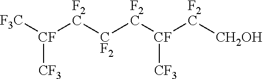

| Molecular | | Molecular | Physical | | Boiling |
|---|---|---|---|---|---|
| Formula | CAS# | Weight | State | Solubility | Point |
| C10H3F19O | 232587-50-7 | 500 | Clear liquid | Insoluble in | 95° C./20 |
| | | | | water | mmHg |

Fluorinated triethylene glycol monobutyl ether, 98%
(1H,1H-Perfluoro-3,6,9-trioxatridecan-1-ol)

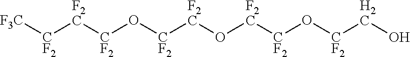 [SMILES: OCC(F)(F)OC(F)(F)C(F)(F)OC(F)(F)C(F)(F)OC(F)(F)C(F)(F)C(F)(F)C(F)(F)F]

| Molecular | | Molecular | Physical | Boiling |
|---|---|---|---|---|
| Formula | CAS# | Weight | State | Point |
| C10H3F19O4 | 317817-24-6 | 548 | Clear liquid | 175-176° C. |

Perfluoro-(2,3-Dimethylbutane), 95%
(1,1,1,2,3,4,4,4-Octafluoro-2,3-bis(trifluoromethyl)butane)

| Molecular | | Molecular | Physical | Boiling | Specific |
|---|---|---|---|---|---|
| Formula | CAS# | Weight | State | Point | Gravity |
| C6F14 | 354-96-1 | 338.04 | Clear liquid | 57-59° C. | 1.7729 |

Perfluoroheptane, 98%
(Hexadecafluoroheptane)

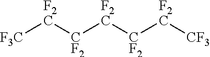 [SMILES: FC(F)(F)C(F)(F)C(F)(F)C(F)(F)C(F)(F)C(F)(F)C(F)(F)F]

| Molecular | | Molecular | Physical | Boiling | Specific | Refractive |
|---|---|---|---|---|---|---|
| Formula | CAS# | Weight | State | Point | Gravity | Index |
| C7F16 | 335- | 388 | Clear | 80-82° C. | 1.72 | 1.269 |
| | 57-9 | | liquid | | | |

Perfluorooctane, 98%
(Octadecafluorooctane)

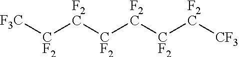 [SMILES: FC(F)(F)C(F)(F)C(F)(F)C(F)(F)C(F)(F)C(F)(F)C(F)(F)C(F)(F)F]

| Molecular | | | Molecular | Physical | Boiling | Melting | Specific | Refractive |
|---|---|---|---|---|---|---|---|---|
| Formula | CAS# | EINECS# | Weight | State | Point | Point | Gravity | Index |
| C8F18 | 307- | 206- | 438 | Clear | 103-104° C. | −25° C. | 1.73 | <1.3000 |
| | 34-6 | 199-2 | | liquid | | | | |

Perfluorononane, 98%
(Eicosafluorononane)

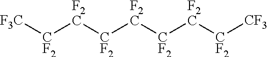 [SMILES: FC(F)(F)C(F)(F)C(F)(F)C(F)(F)C(F)(F)C(F)(F)C(F)(F)C(F)(F)C(F)(F)F]

| Molecular | | Molecular | Physical | Boiling | Melting | Specific | Refractive |
|---|---|---|---|---|---|---|---|
| Formula | CAS# | Weight | State | Point | Point | Gravity | Index |
| C9F20 | 375- | 488 | Clear | 126- | −16° C. | 1.799 | <1.3000 |
| | 96-2 | | liquid | 127° C. | | | |

Perfluorodecalin, 90%
(Octadecafluorodecalin)

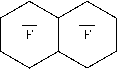

| Molecular | | | Molecular | Physical | Boiling | Melting | Vapor |
|---|---|---|---|---|---|---|---|
| Formula | CAS# | EINECS# | Weight | State | Point | Point | Pressure |
| C10F18 | 306-94-5 | 206-192-4 | 462.10 | Clear liquid | 140° C. | 0° C. | 6 torr @ 20° C. |

1H.4H-Perfluorobutane, 98%
(Octafluorobutane)

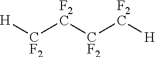 [SMILES: FC(F)C(F)(F)C(F)(F)C(F)F]

| Molecular | | Molecular | Physical | Boiling | Refractive |
|---|---|---|---|---|---|
| Formula | CAS# | Weight | State | Point | Index |
| C4H2F8 | 377-36-6 | 202 | Clear liquid | 45° C. | <1.3000 |

1H-Perfluoropentane, 98%
(Undecafluoropentane)

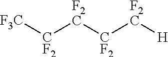 [SMILES: FC(F)C(F)(F)C(F)(F)C(F)(F)C(F)(F)F]

| Molecular | | Molecular | Physical | Boiling | Refractive |
|---|---|---|---|---|---|
| Formula | CAS# | Weight | State | Point | Index |
| C5HF11 | 375-61-1 | 270 | Clear liquid | 42° C. | <1.3000 |

1H,6H-Perfluorohexane, 97%
(Dodecafluorohexane)

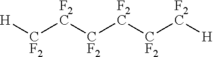 [SMILES: FC(F)C(F)(F)C(F)(F)C(F)(F)C(F)(F)C(F)F]

| Molecular | | Molecular | Physical | Boiling | Refractive |
|---|---|---|---|---|---|
| Formula | CAS# | Weight | State | Point | Index |
| C6H2F12 | 336-07-2 | 302 | Clear liquid | 93° C. | <1.3000 |

1H-Perfluorohexane, 98%
(Tridecafluorohexane)

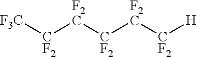 [SMILES: FC(F)C(F)(F)C(F)(F)C(F)(F)C(F)(F)C(F)(F)F]

| Molecular | | Molecular | Physical | Boiling | Refractive |
|---|---|---|---|---|---|
| Formula | CAS# | Weight | State | Point | Index |
| C6HF13 | 355-37-3 | 320 | Clear liquid | 70° C. | <1.3000 |

1H-Perfluoroheptane, 98%
(Pentadecafluoroheptane)

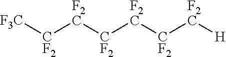 [SMILES: FC(F)C(F)(F)C(F)(F)C(F)(F)C(F)(F)C(F)(F)C(F)(F)F]

| Molecular | | Molecular | Physical | Boiling | Refractive |
|---|---|---|---|---|---|
| Formula | CAS# | Weight | State | Point | Index |
| C7HF15 | 27213-61-2 | 370 | Clear liquid | 97° C. | <1.3000 |

1H,8H-Perfluorooctane, 98%
(Hexadecafluorooctane)

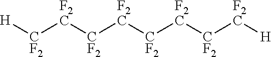 [SMILES: FC(F)C(F)(F)C(F)(F)C(F)(F)C(F)(F)C(F)(F)C(F)(F)C(F)F]

| Molecular | | Molecular | Physical | Boiling | Refractive |
|---|---|---|---|---|---|
| Formula | CAS# | Weight | State | Point | Index |
| C8H2F16 | 307-99-3 | 402 | Clear liquid | 134° C. | <1.3000 |

1H-Perfluorooctane, 98%
(Heptadecafluorooctane)

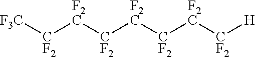 [SMILES: FC(F)C(F)(F)C(F)(F)C(F)(F)C(F)(F)C(F)(F)C(F)(F)C(F)(F)F]

| Molecular | | Molecular | Physical | Boiling | Refractive |
|---|---|---|---|---|---|
| Formula | CAS# | Weight | State | Point | Index |
| C8HF17 | 335-65-9 | 420 | Clear liquid | 117° C. | <1.3000 |

1H-Perfluorononane, 98%
(Nonadecafluorononane)

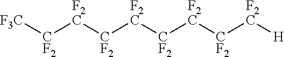 [SMILES: FC(F)C(F)(F)C(F)(F)C(F)(F)C(F)(F)C(F)(F)C(F)(F)C(F)(F)C(F)(F)F]

| Molecular | | Molecular | Physical | Boiling | Refractive |
|---|---|---|---|---|---|
| Formula | CAS# | Weight | State | Point | Index |
| C9HF19 | 375-94-0 | 470 | Clear liquid | 138° C. | <1.3000 |

Perfluorodiglyme, 98%
(Perfluoro(diethylene glycol dimethyl ether))

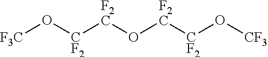 [SMILES: FC(F)(F)OC(F)(F)C(F)(F)OC(F)(F)C(F)(F)OC(F)(F)F]

| Molecular | | Molecular | Physical | Boiling | Refractive |
|---|---|---|---|---|---|
| Formula | CAS# | Weight | State | Point | Index |
| C6F14O3 | 40891-99-4 | 386 | Clear liquid | 66° C. | <1.3000 |

Perfluorotriglyme, 98%
(Perfluoro(triethylene glycol dimethyl ether))

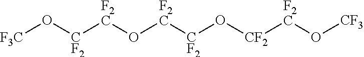

| Molecular | | Molecular | Physical | Boiling | Refractive |
|---|---|---|---|---|---|
| Formula | CAS# | Weight | State | Point | Index |
| C8F18O4 | 64028-04-2 | 502 | Clear liquid | 106° C. | <1.3000 |

Perfluorotetraglyme, 98%
(Perfluoro(triethylene glycol dimethyl ether))

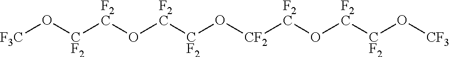

| Molecular | | Molecular | Physical | Boiling | Refractive |
|---|---|---|---|---|---|
| Formula | CAS# | Weight | State | Point | Index |
| C10F22O5 | 64028-6-4 | 618 | Clear liquid | 138° C. | <1.3000 |

Perfluoro-[15]-crown-5 ether, 99%
(Eicosafluoro-[15]-crown-5 ether)

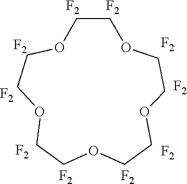

| Molecular | | Molecular | Physical | Boiling | Specific |
|---|---|---|---|---|---|
| Formula | CAS# | Weight | State | Point | Gravity |
| C10F20O5 | 97571-69-2 | 580 | Clear liquid | 145° C. | 1.78 |

| | |
|---|---|
| Commercial name | Boiling Point |
| HFA 134a ™ | −26 |
| Commercial name | Boiling Point |
| HFA227ea ™ | −17 |

### Other Perfluorinated Liquids and Their Boiling Points

| | Boiling point |
|---|---|
| Perfluorinated liquids | ° C. |
| perfluoropropane | −36.7 |
| Perfluorobutane | −1.7 |
| perfluoropentane | 28.0 |
| Perfluorohexane | 56.0 |
| perfluoroheptane | 56.0 |
| methyl perfluoropropyl | 34 |
| ether | |
| Perfluoroperhydrophenanthrene | 215 |
| Perfluorooctylbromide | 142 |
| perfluoro tributyl amine | 178 |
| perfluorotripentyl amine | 215 |
| methyl perfluorobutylether | 60 |
| 2,2,2-trifluoroethanol | 78 |

### Pharmaceutical Compositions

Below is a description of pharmaceutical compositions comprising fluorinated and or/perfluorinated liquid, therapeutics e.g. corticosteroid or vasodilator or antibiotic or antiviral drug and optional concentration of oxygen. The compositions can optionally deliver oxygen and therapeutics to alveolar membrane and reduces alveolar inflammations when necessary. Method of preparation of pharmaceutical compositions is described. Method of treatment is described.

The present disclosure describes and provides for the treatment of subjects suffering from pneumonia, bronchiolitis, asthma, COPD or other diseases and/or conditions of the pulmonary tissue. In one aspect the present disclosure relates to a treatment of a mammal, especially human, suffering from viral pneumonia. In another aspect the present disclosure relates to a treatment of a mammal, especially human, suffering from bacterial pneumonia. In another aspect the present disclosure relates to a treatment of a mammal, especially human, suffering from asthma or COPD. Yet in another aspect the present disclosure relates to a prophylactic treatment of a mammal, especially a human, suffering from chronic asthma or COPD that may be exposed to a viral pandemic or community infection that is defined as the spread of infection via social interactions.

The treatments described herein include those in which a fluorinated and/or perfluorinated liquids is administered to one or more lung tissues via inhalation. The treatments can optionally be employed to deliver dissolved oxygen and therapeutically effective dose of various APIs such as corticosteroids and/or vasodilators (e.g, prostacyclin, albuterol). The treatments can also be used to administer antibiotic or antiviral APIs alone or in combination with other API's such as corticosteroids. Delivery of the API(s) can be in the form of aerosolized, nebulized, and/or gaseous (volatilized) API's administered concurrently (e.g., separately or as an admixture) or administered sequentially.

Since the density of fluorinated and/or perfluorinated liquids is twice the density of water, a liquid fluorocarbon droplet sinks through alveolar edema under the influence of gravity. One realization of the present disclosure delivers therapeutics (e.g. corticosteroids or antibiotics or anti-viral drugs) and dissolved oxygen via with fluorinated and/or perfluorinated liquids at optional concentration to alveolar tissue across the fluid pneumonia layer.

An aspect of the current disclosure is directed to compositions, methods of preparing, compositions, and the therapeutic use of compositions comprising fluorinated liquids, perfluorinated liquids, and mixtures thereof having low surface tension (9.5 mN sec⁻¹) and the boiling point between 27° C.-45° C., and will be exhaled fast, loosening the phlegm from the airways. Accordingly, the present disclosure includes and provides for compositions, methods of preparing, compositions, and the therapeutic use of compositions comprising fluorinated and/or perfluorinated liquids and having the boiling point between 37° C.-45° C., alone or in combination with one or more APIs, for use in loosening phlegm and as an expectorant.

In an aspect, the current disclosure includes and provides for a corticosteroid or prostacyclin formulated with one or more fluorinated and/or perfluorinated liquid shows increased anti-inflammatory action at lower corticosteroid or prostacyclin dose, and/or synergistic interactions with the reduction in inflammation resulting from administration of perfluorinated liquids. Accordingly, the present disclosure includes and provides for compositions, methods of preparing, compositions, and the therapeutic use of compositions comprising a corticosteroid or prostacyclin formulated with one or more fluorinated and/or perfluorinated liquid.

In an aspect, the current disclosure includes and provides for the use of one or more fluorinated and/or perfluorinated liquids alone or in combination to suppress the overactive immune response and/or cytokine storm that arises during bouts of asthma or during viral or bacterial infection of the alveoli. Accordingly, the present disclosure includes and provides for compositions, methods of preparing, compositions, and the therapeutic use of compositions comprising one or more fluorinated and/or perfluorinated liquids for the suppression of an overactive immune response and/or cytokine storm. The compositions comprising one or more fluorinated and/or perfluorated liquids can that optionally comprise one or more APIs (e.g., one or more corticosteroids and/or prostacyclins). Such compositions find particularly use in treatment of diseases, disorders, and/or conditions involving the lungs or a pulmonary tissue.

The present disclosure includes and provides for therapeutic compositions comprising one or more APIs (e.g., corticosteroids, prostacyclins, antibiotics and/or antiviral drugs) that can be combined with a fluorinated and/or per fluorinated liquid by reverse emulsification using a fluorinated surfactant and one or more cosolvents. Accordingly, the disclosure encompasses both a method of preparing such reverse emulsions, the reverse emulsions, and the therapeutic use of the reverse emulsion (e.g., as an inhaled therapeutic composition) for treatment of one or more disease, disorders, and/or conditions affecting the lungs or pulmonary tissues. In one embodiment, a fluorinated and/or perfluorinated liquid composition is an emulsion is mixed with a preformed corticosteroid; prostacyclin/anti-virus drug emulsion to form a therapeutic composition.

Another aspect of the present disclosure is directed to nano (1-100 nm) and/or micro (0.1 μm-10 μm) particles of one or more APIs (e.g., one or more corticosteroids, prostacyclins, antibiotics, and/or antiviral drugs) that are uniformly suspended in fluorinated and/or perfluorinated liquid composition using a fluorinated surfactant. The present disclosure includes and provides for methods of preparing such compositions, and the therapeutic use of such compositions for the treatment of one or more diseases, disorders, and/or conditions affecting the lungs or a pulmonary tissue.

The present disclosure includes and provides for compositions, methods of preparing, compositions, and the therapeutic use of compositions comprising one or more of a vasodilator, antibiotic, and/or antiviral drug dissolved in a hydrophilic solvent emulsified with a fluorinated and/or perfluorinated liquid composition in the presence of a surfactant,

In one embodiment corticosteroid/prostacyclin/anti-virus drug is dissolved in fluorinated and/or perfluorinated liquids using an alcohol as cosolvents.

In one embodiment nitric oxide is dissolved in deoxygenated fluorinated and/or perfluorinated liquids.

### 1.1 Formulations

### Corticosteroid

Corticosteroids are a class of steroid hormones that are produced in the adrenal cortex of vertebrates, as well as the synthetic analogues of these hormones. Two main classes of corticosteroids, glucocorticoids and mineralocorticoids, are involved in a wide range of physiological processes, including stress response, immune response, and regulation of inflammation, carbohydrate metabolism, protein catabolism, blood electrolyte levels, and behavior. Inhaled corticosteroids are used for treating asthma attack and respiratory distress syndrome. In the present disclosure, the mixture consisting of fluorinated and/or perfluorinated liquids, dissolved oxygen at an optional concentration and a corticosteroid will penetrate across the pneumonia layer and deliver corticosteroid and oxygen to alveolar membrane. Corticosteroid and fluorinated and/or perfluorinated liquids will suppress inflammatory response in pulmonary tissue and loosen phlegm when volatile fluorinated and/or perfluorinated liquid are exhaled.

The embodiment where corticosteroid solution in an alcohol is reverse emulsified in fluorinated and/or perfluorinated liquids, the mixture of fluorinated and/or perfluorinated liquids will carry the corticosteroid dissolved in the alcohol microdroplet stabilized by a fluorinated surfactant. Corticosteroid solution will be absorbed by alveolar membrane. Fluorinated and/or perfluorinated liquids will be partially absorbed by alveolar membrane along with dissolved oxygen which will diffuse through the membrane. Diffused oxygen will oxygenate blood and fluorinated and/or perfluorinated liquids will also reduce the inflammatory responses. Un absorbed fluorinated and/or perfluorinated liquids will be exhaled which will loosen the phlegm from the airways.

The embodiment where corticosteroid emulsion and an emulsion of fluorinated and/or perfluorinated liquids are mixed together, the mixture of fluorinated and/or perfluorinated liquids will help the corticosteroid emulsion to reach the alveolar membrane. Fluorinated and/or perfluorinated liquids will be partially absorbed by alveolar membrane along with dissolved oxygen which will diffuse through the membrane. Diffused oxygen will oxygenate blood and fluorinated and/or perfluorinated liquids will reduce the inflammatory responses. Unabsorbed fluorinated and/or perfluorinated liquids will be exhaled which will loosen the phlegm from the airways. Opened up airways will allow corticosteroid emulsion to effectively reach the alveolar membrane.

The embodiment where corticosteroid nano/micro particles are uniformly dispersed in fluorinated and/or perfluorinated liquids using a fluorinated surfactant, the mixture of fluorinated and/or perfluorinated liquids will help the corticosteroid nano/micro particles to reach the alveolar membrane and get slowly absorbed by alveolar membrane over the period of time. Fluorinated and/or perfluorinated liquids will be partially absorbed by alveolar membrane along with dissolved oxygen which will diffuse through the membrane. Diffused oxygen will oxygenate blood and fluorinated and/or perfluorinated liquids will also reduce the inflammatory responses. Unabsorbed fluorinated and/or perfluorinated liquids will be exhaled which will loosen the phlegm from the airways. Opened up airways will allow corticosteroid nano/micro particles to effectively reach the alveolar membrane. Corticosteroid nano/micro particles will be absorbed slowly through alveolar membrane suppressing the inflammatory response for prolonged period of time.

The embodiment where corticosteroid is dissolved in the mixture of fluorinated and/or perfluorinated liquids with the help of an alcohol cosolvent, the mixture of fluorinated and/or perfluorinated liquids will help the corticosteroid to reach the alveolar membrane and get readily absorbed. Fluorinated and/or perfluorinated liquids will also be partially absorbed by alveolar membrane along with dissolved oxygen which will diffuse through the membrane. Diffused oxygen will oxygenate blood and fluorinated and/or perfluorinated liquids and corticosteroid will also reduce the inflammatory responses. Unabsorbed fluorinated and/or perfluorinated liquids will be exhaled which will loosen the phlegm from the airways facilitating the natural breathing process.

### Antibiotic

Inhaled antibiotics have been used to treat chronic airway infections since the 1940s. Antibiotics currently marketed for inhalation include nebulized and dry powder forms of tobramycin and colistin and nebulized aztreonam. tobramycin designed for inhalation was approved by the U.S. Food and Drug Administration (FDA) for use in subjects with cystic fibrosis (CF) with chronic Pseudomonas aeruginosa infection. In the present disclosure, the mixture consisting of fluorinated and/or perfluorinated liquids, dissolved oxygen at an optional concentration and an antibiotic will penetrate across the pneumonia layer and deliver antibiotics and oxygen to alveolar membrane, reduce bacterial infection, suppress inflammatory response in pulmonary tissue and loosen phlegm when volatile fluorinated and/or perfluorinated liquids are exhaled.

The embodiment where antibiotic is dissolved in water and fluorinated and/or perfluorinated liquids is emulsified in that antibiotic solution with a surfactant, the mixture of fluorinated and/or perfluorinated liquids opens up the airways and help the antibiotic to reach the alveolar membrane. Fluorinated and/or perfluorinated liquids are partially absorbed by alveolar membrane along with dissolved oxygen which diffuses through the membrane. Diffused oxygen will oxygenate blood and the mixture of fluorinated and/or perfluorinated liquids will also reduce the inflammatory responses. Unabsorbed fluorinated and/or perfluorinated liquids will be exhaled which will loosen the phlegm from the airways. Opened up airways will allow antibiotic to effectively reach the alveolar membrane and heal bacterial infection.

### Anti Virus Drug

Systemic administration of antivirus drugs is a standard practice for the treatment of deadly viral infections. Remdesivir which has chemical name 2-Ethylbutyl (2S)-2-{[(S)-{[(2R,3S,4R,5R)-5-(4-aminopyrrolo[2,1-f][1,2,4]triazin-7-yl)-5-cyano-3,4-dihydroxytetrahydrofuran-2-yl]methoxy}(phenoxy)phosphoryl]amino}propanoate is used for the treatment of viral pneumonia COVID-19. In the present disclosure, the mixture consisting of fluorinated and/or perfluorinated liquids, dissolved oxygen at an optional concentration and an antivirus drug will penetrate across the pneumonia layer and deliver anti-virus drug and oxygen to alveolar membrane. Fluorinated and/or perfluorinated liquids will suppress inflammatory response in pulmonary tissue and loosen phlegm when volatile fluorinated and/or perfluorinated liquids are exhaled.

The embodiment where anti-virus drug is first dissolved in an alcohol then reverse emulsified in fluorinated and/or perfluorinated liquids, the mixture of fluorinated and/or perfluorinated liquids will caily the anti-virus drug dissolved in the alcohol microdroplet stabilized by a fluorinated surfactant. The anti-virus drug solution will be absorbed by alveolar membrane. Fluorinated and/or perfluorinated liquids will be partially absorbed by alveolar membrane along with dissolved oxygen which will diffuse through the membrane. Diffused oxygen will oxygenate blood and fluorinated and/or perfluorinated liquids will also reduce the inflammatory responses. Un absorbed fluorinated and/or perfluorinated liquids will be exhaled which will loosen the phlegm from the airways.

The embodiment where anti-virus drug emulsion and an emulsion of fluorinated and/or perfluorinated liquids are mixed together, the mixture of fluorinated and/or perfluorinated liquids will help the anti-virus drug emulsion to reach the alveolar membrane. Fluorinated and/or perfluorinated liquids will be partially absorbed by alveolar membrane along with dissolved oxygen which will diffuse through the membrane. Diffused oxygen will oxygenate blood and fluorinated and/or perfluorinated liquids will reduce the inflammatory responses. Unabsorbed fluorinated and/or perfluorinated liquids will be exhaled which will loosen the phlegm from the airways. Opened up airways will allow anti-virus drug emulsion to effectively reach the alveolar membrane.

The embodiment where anti-virus drug nano/micro particles are uniformly dispersed in fluorinated and/or perfluorinated liquids using a fluorinated surfactant, the mixture of fluorinated and/or perfluorinated liquids will help the anti-virus drug nano/micro particles to reach the alveolar membrane and get slowly absorbed by alveolar membrane over the period of time. Fluorinated and/or perfluorinated liquids will be partially absorbed by alveolar membrane along with dissolved oxygen which will diffuse through the membrane. Diffused oxygen will oxygenate blood and fluorinated and/or perfluorinated liquids will also reduce the inflammatory responses. Unabsorbed fluorinated and/or perfluorinated liquids will be exhaled which will loosen the phlegm from the airways. Opened up airways will allow anti-virus drug nano/micro particles to effectively reach the alveolar membrane. The anti-virus drug nano/micro particles will be absorbed slowly through alveolar membrane suppressing the inflammatory response for prolonged period of time.

The embodiment where anti-virus drug is dissolved in the mixture of fluorinated and/or perfluorinated liquids with the help of an alcohol cosolvent, the mixture of fluorinated and/or perfluorinated liquids will help the anti-virus drug to reach the alveolar membrane and get readily absorbed. Fluorinated and/or perfluorinated liquids will also be partially absorbed by alveolar membrane along with dissolved oxygen which will diffuse through the membrane. Diffused oxygen will oxygenate blood and fluorinated and/or perfluorinated liquids will reduce the inflammatory responses. Unabsorbed fluorinated and/or perfluorinated liquids will be exhaled which will loosen the phlegm from the airways facilitating the natural breathing process.

### Vasodilator

Inhaled pulmonary vasodilators including nitric oxide, aerosolized prostacyclin, and jet nebulized salbutamol are used for treating severe refractory hypoxemia in subjects with asthma and acute respiratory distress syndrome. In the current disclosure, the mixture consisting of fluorinated and/or perfluorinated liquids, dissolved oxygen at an optional concentration and a vasodilator, relaxes muscles in the airways, reduce inflammatory response and deliver oxygen to alveolar membrane. When volatile fluorinated and/or perfluorinated liquids will be exhaled it will loosen phlegm helping airways to open up further for the natural breathing.

The embodiment where a therapeutic dose of a vasodilator e.g. salbutamol is dissolved in water and a mixture of fluorinated and/or perfluorinated liquids is emulsified in that salbutamol solution using a surfactant, the vasodilator relaxes the muscles in the airways and allows the mixture of fluorinated and/or perfluorinated liquids to reach the alveolar membrane. Fluorinated and/or perfluorinated liquids will be partially absorbed by alveolar membrane along with dissolved oxygen which will diffuse through the membrane. Diffused oxygen will oxygenate blood and fluorinated and/or perfluorinated liquids will also reduce the inflammatory responses. Unabsorbed fluorinated and/or perfluorinated liquids will be exhaled which will loosen the phlegm helping airways to open up further for the natural breathing.

The embodiment where prostacyclin solution in an alcohol is reverse emulsified in fluorinated and/or perfluorinated liquids using a fluorinated surfactant, the mixture of fluorinated and/or perfluorinated liquids will carry prostacyclin dissolved in the alcohol microdroplet stabilized by a fluorinated surfactant. Prostacyclin solution will be absorbed by vascular wall relax the smooth muscle and allow fluorinated and/or perfluorinated liquids to reach alveolar membrane. Fluorinated and/or perfluorinated liquids will be partially absorbed by alveolar membrane along with dissolved oxygen which will diffuse through the membrane. Diffused oxygen will oxygenate blood and fluorinated and/or perfluorinated liquids will also reduce the inflammatory responses. Un absorbed fluorinated and/or perfluorinated liquids will be exhaled which will loosen the phlegm from the airways.

The embodiment where prostacyclin emulsion and an emulsion of fluorinated and/or perfluorinated liquids are mixed together, the prostacyclin emulsion will help the mixture of fluorinated and/or perfluorinated liquids to reach the alveolar membrane. Prostacyclin absorbed by vascular membrane will relax smooth muscles and opens up the airways. Fluorinated and/or perfluorinated liquids will be partially absorbed by alveolar membrane along with dissolved oxygen which will diffuse through the membrane. Diffused oxygen will oxygenate blood and fluorinated and/or perfluorinated liquids will reduce the inflammatory responses. Unabsorbed fluorinated and/or perfluorinated liquids will be exhaled which will loosen the phlegm from the airways facilitating the natural breathing.

### Gaseous Therapeutics

The mixture consisting of deoxygenated fluorinated and/or perfluorinated liquids and nitric oxide as a gaseous vasodilator, relaxes muscles in the airways and reduce inflammatory response in alveoli.

The embodiment where a therapeutic dose of nitric oxide is dissolved in fluorinated and/or perfluorinated liquids, the nitric oxide relaxes the muscles in the airways and allows the mixture of fluorinated and/or perfluorinated liquids to reach the alveolar membrane. Fluorinated and/or perfluorinated liquids will be partially absorbed by alveolar membrane and reduce the inflammatory responses. Unabsorbed fluorinated and/or perfluorinated liquids will be exhaled which will loosen the phlegm helping airways to open up further for the natural breathing.

### 1.1.1 Reverse Emulsion

Reverse emulsification is a technique by which a small volume of a hydrophilic solution is dispersed in hydrophobic bulk solvent with the help of a surfactant. In the present disclosure, corticosteroid or antibiotics or anti-virus drug or vasodilator will be dissolved in a hydrophilic solvent, a mixture of fluorinated and/or perfluorinated liquids will be used as the hydrophobic bulk liquid and partially fluorinated molecule or block copolymers will be used as fluorinated surfactant. Hydrophilic solvent can include but not limited to alcohol, ketone, ether; polyether, amine, amide or ester. The fluorinated and/or perfluorinated liquids can include, but is not limited to, one or more selected from: perfluoropropane, perfluorobutane, perfluoropentane, perfluorohexane, perfluorooctane, perfluorodecalin, perfluoroperhydrophenanthrene, perfluorooctylbromide, perfluoro tributyl amine; perfluorotripentyl amine, poiy(hexafluoropropylene oxide) and combinations thereof. Fluorinated surfactant used can include but not limited to polyethylene glycol-co-perfluropolyethylene oxide, or polyethylene gycol-co-perfluoropolypropylene oxide, or polypropyleneoxide-co-perfluoropolyethylene oxide or polypropyl eneoxide-co-perfluoropollypropylene oxide polyglycolide-co-perfluoropolypropylene oxide or polyglycolide-co-perfluoropolyethylene oxide or perfluoropolypropylene oxide conjugated phospholipids or perfluoroalkyl conjugated phospholipids.

In an embodiment where the hydrophilic solution is dispersed in the mixture of fluorinated and/or perfluorinated liquids, the hydrophilic solvent constitutes about 2-5%, about about 10-20%, about 20-30%, about 30-40% or about 40-50% of the total dispersion on a weight basis.

In an embodiment where the mixture of fluorinated and/or perfluorinated liquids is used as the bulk liquid phase, it constitutes about about 50%, about 60%, about 70%, about 80%, about 90%, about 95%, about 98% of the total dispersion on a weight basis.

In an embodiment where a hydrophilic solution of corticosteroid is dispersed in the mixture of the fluorinated and/or perfluorinated liquids, the concentration of corticosteroid ranges from about 5-10 mcg/mL, about 10-15, mcg/mL, about 15-20 mcg/mL, about 20-25 mcg/mL, about 25-30 mcg/mL, about 30-35 mcg/mL, about 35-40 mcg/mL, about 40-45 mcg/mL, or about 45-50 mcg/mL.

In an embodiment where a hydrophilic solution of antibiotic is dispersed in the mixture of the fluorinated and/or perfluorinated liquids, the concentration of antibiotic in the final dispersion ranges from about 5-10 mg/mL, about 10-20 mg/mL, about 20-30 mg/mL, about 30-40 mg/mL, about 40-50 mg/mL, about 50-100 mg/mL.

In an embodiment where a hydrophilic solution of an antivirus drug is dispersed in the mixture of the fluorinated and/or perfluorinated liquids, the concentration of anti-virus drug in the final dispersion ranges from about 5-10 mg/mL, about 10-20 mg/mL, about 20-30 mg/nL, about 30-40 mg/mL, about 40-50 mg/mL, about 50-100 mg/mL.

In an embodiment where a hydrophilic solution of vasodilator is dispersed in the mixture of the fluorinated and/or perfluorinated liquids, the concentration of vasodilator in the final dispersion ranges from about 0.1-0.5 mg/mL, about 0.5-1.0 mg/mL, about 1.0-1.5 mg/mL, about 1.5-2.0 mg/mL.

In an embodiment where a fluorinated surfactant is used to stabilize the dispersion, the range of fluorinated surfactant concentration in the final dispersion is about 0.5-1%, about 1-2%, about 2-3%, about 3-4%, about 4-5%, about 5-6%, about 6-7%, about 7-8?, about 8-9% or about 9-10% on a weight basis.

In an embodiment where a hydrophilic solution is dispersed in fluorinated and/or perfluorinated liquids using a fluorinated surfactant, the particle size of dispersed hydrophilic solution ranges from about 5-10 nm, about 10-30 nm, about 30-300 nm, about 300-500nm, about 500-750 nm, about 750 nm-1 μm, about 1-10 μm as determined by dynamic light scattering technique.

In an embodiment where a hydrophilic solution of corticosteroid or antibiotic or vasodilator is dispersed in the mixture of fluorinated and/or perfluorinated liquids with the help of a fluorinated surfactant, the dispersion remain stable (not more than 5% change in particle size) for about 30 min-1 hour, about 1-2 hour, about 2-3 hour, about 3-4 hour, about 4-12 hour, about 12-48 hour, about 48-96 hour. In some embodiments, the dispersion is uniformly distributed upon shaking.

In an embodiment about 2%-50% hydrophilic cosolvent, about 50-98% fluorinated and/or perfluorinated liquid mixture and about 0.5-10% fluorosurfactant is used to disperse an. API which can include but not limited to vasodilator or antivirus drug or antibiotic or corticosteroid.

### 1.1.2 Mixtures of Emulsions

In the present disclosure an emulsion of the fluorinated and/or perfluorinated liquids compounds is mixed with another emulsion of corticosteroid or prostacyclin or antivirus drug to produce the mixture of emulsions where two separately emulsified micro/nano droplets will independently coexist or coalesce into a single micro/nano droplet. The electrostatic charge of the surfactant used for the individual emulsions are both cationic, anionic or neutral. The fluorinated and/or perfluorinated liquids can include, but is not limited to, one or more selected from: perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide) and combinations thereof. Surfactants used can include but not limited to natural lipids listed in table 3, especially phospholipids, phosphoglycolipids, synthetic phospholipids, phospholipid polyethylene glycol conjugate, poly lactide-co-glycolide and their combinations.

In an embodiment where the emulsions of fluorinated and/or perfluorinated liquids is mixed with another emulsion of corticosteroid or prostacyclin, the fluorinated and/or perfluorinated liquids compounds essentially constitute greater than about 5%, about 10%, about about 30%, about 40%, about 50% of the final mixture on a weight basis.

In an embodiment where the corticosteroid or prostacyclin or antivirus drug is dissolved in a hydrophilic solvent before making the emulsion, the hydrophilic solvent can include but not limited to alcohol, ketone, ether, polyether, amine, amide or ester. The concentration of cosolvent in the final mixture is less than about 1.0%, about 2%, about 5%, about 10%, about 15%, about 20%.

In an embodiment where the individual emulsions are prepared before mixing together, the concentration of the surfactant used in either emulsion is less than about 0.1%, about 0.5%, about 1.0%, about 2.0%, about 5.0%.

In an embodiment the emulsion of fluorinated and/or perfluorinated liquids compounds and the corticosteroid or prostacyclin emulsion are mixed at the point of care right before the delivery to subject's pulmonary tissue. Such mixture of two emulsion is stable for more than about 2 minutes, about 10 minutes, about 30 minutes, about 60 minutes or about 90 minutes. In case of longer storage after mixing the emulsions, the average particle size of the emulsion changes>5% it becomes uniform upon shaking.

In an embodiment where two emulsions are previously mixed before the packaging, the emulsion mixture is stable for more than about 30 days, about 6 months, about 1 year or about 2 years. In case of longer storage, the average particle size of the emulsion changes>5% it becomes uniform upon shaking.

1.1.3 Fluorinated and/or Perfluorinated Liquids Emulsion in Antibiotic or Vasodilator Solution

In the present disclosure the mixture of fluorinated and/or perfluorinated liquids compounds is emulsified with the help of surfactants in a hydrophilic solution of antibiotics or vasodilator. The fluorinated and/or perfluorinated liquids can include, but is not limited to, one or more selected from: perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide) and combinations thereof. Hydrophilic solvent can include but not limited to water, ethanol, isopropanol, butanol, isobutanol, or a combination thereof with optional concentration of inorganic salts. Surfactants used can include but not limited to natural lipids listed in table 3, especially phospholipids, phosphoglycolipids, synthetic phospholipids, phospholipid-polyethylene glycol conjugate, poly lactide-co-glycolide and their combinations.

### Micro/Nanoparticle Suspensions

In the present disclosure micro/nanoparticles of corticosteroid or prostacyclin or antibiotic or anti-virus drug are suspended in a mixture of fluorinated and/or perfluorinated liquids compounds with the help of a fluorinated surfactant. The fluorinated and/or perfluorinated liquids can include, but is not limited to, one or more selected from: perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide) and combinations thereof. Fluorinated surfactant used can include but not limited to polyethylene glycol-co-perfluropolyethylene oxide, or polyethylene gycol-co-perfluoropolypropylene oxide, or polypropyleneoxide-co-perfluoropolyethylene oxide or polypropyleneoxi de-co-perfluoropolypropylene oxide, polyglycolide-co-perfluoropolypropylene oxide or polyglycolide-co-perfluoropolyethylene oxide or perfluoropolypropylene oxide conjugated phospholipids or perfluoroalkyl conjugated phospholipids.

In an embodiment where corticosteroid or prostacyclin micro/nanoparticles are suspended in fluorinated and/or perfluorinated liquids, the concentration of corticosteroid or prostacyclin in the final suspension ranges from about 5-10 mcg/mL, about 10-15, mcg/mL, about 15-20 mcg/mL, about 20-25 mcg/mL, about 25-30 mcg/mL, about 30-35 mcg/mL, about mcg/mL, about 40-45 mcg/mL or about 45-50 mcg/mL.

In an embodiment where antibiotic/anti-virus drug micro/nanoparticles are suspended in fluorinated and/or perfluorinated liquids the concentration of antibiotic in the final suspension ranges from about 5-10 mg/mL, about 10-20 mg/mL, about 20-30 mg/mL, about 30-40 mg/mL, about 40-50 mg/mL, about 50-100 mg/mL.

In an embodiment where antibiotic or corticosteroid or prostacyclin micro/nanoparticles are suspended in fluorinated and/or perfluorinated liquids compounds with the help of a fluorinated surfactant, the fluorinated surfactant concentration in the final suspension ranges from about 0.1-0.5%, about 0.5-1.0%, about 2.0-5.0%.

In an embodiment 0.1-5% fluorinated surfactants are used to disperse an active pharmaceutical ingredient into a mixture of fluorinated and/or perfluorinated liquids which constitutes about 95-99% of the total formulation on a weight basis.

### 1.1.4 Solutions

In the present disclosure, corticosteroid or prostacyclin or anti-virus drug is dissolved in a mixture of fluorinated and/or perfluorinated liquids compounds with the help of cosolvents. The fluorinated and/or perfluorinated liquids can include, but is not limited to, one or more selected from: perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), hydrofluoroalkane (e.g. 1H,4H-perfluorobutane, 1h-perfluoropentane, HFA 134a™, HFA227ea™), hydrofluoroether (e.g.methyl perfluorobutylether, methyl perfluoropropyl ether (3M Novec 7000™)), hydrofluoro alcohols (e.g. 2,2,2-trifluoroethanol) and combinations thereof.

In an embodiment where corticosteroid or prostacyclin is dissolved in fluorinated and/or perfluorinated liquids with the help of cosolvents, the concentration of corticosteroid or prostacyclin in the final suspension ranges from about 5-10 mcg/mL, about 10-15, mcg/mL, about 15-20 mcg/mL, about 20-25 mcg/mL, about 25-30 mcg/mL, about 30-35 mcg/mL, about mcg/mL, about 40-45 mcg/mL or about 45-50 mcg/mL.

In an embodiment where anti-virus drug is dissolved in fluorinated and/or perfluorinated liquids with the help of cosolvents, the concentration of anti-virus drug in the final suspension ranges from about 5-10 mg/mL, about 10-20 mg/mL, about 20-30 mg/mL, about 30-40 mg/mL, about 40-50 mg/mL, about 50-100 mg/mL.

In an embodiment where cosolvents are used to dissolve corticosteroid or prostacyclin in the mixture of fluorinated and/or perfluorinated liquids compounds, the cosolvent can include but is not limited to one or more alcohols (e.g. ethanol, propanol, isopropanol, butanol, isobutanol), The concentration of cosolvents in the final solution ranges from about 1-10%, about 10-15%, about 15-20%, about 20-30%, about 30-35%, about 35-40%, about 40-45%, about 45-50%.

### 1.2 In an Embodiment about 1-50% Cosoivent is Used on a Weight Basis to Dissolve corticosteroid or prostacyclin in the Mixture of Fluorinated and/or Perfluorinated Liquids which Constitutes About 50-99% of the Final Composition on a Weight Basis

### Corticosteroids

In the present disclosure the corticosteroid mixed with fluorinated and/or perfluorinated liquids compounds in the form of reverse emulsion or mixture of emulsion or micro/nano particle suspension or a solution, can include but is not limited to one or more compounds from the groups listed in Table 3.

TABLE 3
List of corticosteroids

| | | Long chain/ | |
|---|---|---|---|
| | | esterified or | |
| Fluorinated | Hydroxyl containing | cyclic acetals | Glucocorticoseroid |
| Diflorasone | Alclometasone | Desonide | Rimexolone |
| Dexamethasone | Tixocortol | Deflazacort | beclomethasone |
| Desoximetasone | Cloprednol | Ciclesonide | Cortisone |
| Clocortolone | Chloroprednisone | Budesonide | hydrocortisone |
| Clobetasone | Medrysone | Mometasone | Methylprednisolone |
| Clobetasol | Progesterone | Beclometasone | Prednisolone |
| Betamethasone | Pregnenolone | Prednicarbate | Prednisone |
| Fluperolone | Cortisol | Difluprednate | Triamcinolone |
| Fludrocortisone | Corticosterone | Prebediolone | |
| | | acetate | |
| Flugestone | Aldosterone | Loteprednol | |
| Fluorometholone | 21-Hydroxypregnenolone | Cortivazol | |
| Fluprednisolone | 21-Deoxycortisone | RU-28362 | |
| Diflucortolone | 21-Deoxycortisol | | |
| Fluclorolone | 18-Hydroxyprogesterone | | |
| Flumetasone | 18-Hydroxycorticosterone | | |
| Fluocortin | 18-Hydroxy-11- | | |
| | deoxycorticosterone | | |
| Fluocortolone | 17α-Hydroxyprogesterone | | |
| Fluprednidene | 17α-Hydroxypregnenolone | | |
| Fluticasone | 17α,21- | | |
| | Dihydroxypregnenolone | | |
| Fluticasone furoate | 11-Dehydrocorticosterone | | |
| Halometasone | 11-Deoxycorticosterone | | |
| Paramethasone | 11-Deoxycortisol | | |
| Ulobetasol | 11-Ketoprogesterone | | |
| Amcinonide | 11β-Hydroxypregnenolone | | |
| Formocortal | 11β-Hydroxyprogesterone | | |
| Fluclorolone | 11β,17α,21- | | |
| acetonide | Trihydroxypregnenolone | | |
| Fludroxycortide | Meprednisone | | |
| Flunisolide | Prednylidene | | |
| Fluocinolone | | | |
| acetonide | | | |
| Fluocinonide | | | |
| Halcinonide | | | |
| Triamcinolone | | | |
| acetonide | | | |

Corticosteroids listed under hydroxyl containing corticosteroid are more suitable for mixing with fluorinated and/or perfluorinated liquids compounds by reverse emulsification technique. This is due to their high solubility in alcohol, ketone, ether, polyether, amine, amide or ester solvents. Corticosteroids under Long chain/esterified or cyclic acetals are more suitable for mixing with fluorinated and/or perfluorinated liquids in nano/microparticle form. Corticosteroids listed under fluorinated corticosteroids are more suitable for solution phase mixing with the mixture of fluorinated and/or perfluorinated liquids compounds.

### Antibiotics

In the present disclosure antibiotics mixed with fluorinated and/or perfluorinated liquids compounds in the form of reverse emulsion or dissolved in the hydrophilic phase of fluorinated and/or perfluorinated liquids emulsion/suspension as microlnano particle, can include but are not limited to one or more of the following antibiotics: Colistin, Tobramycin, Amikacin, Amphotericin B, Ceftazidime, Gentamicin.

### Vasodilator

In the present disclosure vasdilators mixed with fluorinated and/or perfluorinated liquids compounds in the form of reverse emulsion or dissolved in the hydrophilic phase of fluorinated and/or perfluorinated liquids emulsion or dissolved in fluorinated and/or perfluorinated liquids or suspended as micro/nano particle in fluorinated and/or perfluorinated liquids, can include but is not limited to one or more of the following vasodilator: nitric oxide, salbutamol, prostacyclin.

### Anti-Virus Drug:

In the present disclosure the anti-virus drug mixed with fluorinated and/or perfluorinated liquids compounds in the form of reverse emulsion or mixture of emulsion or micro/nano particle suspension or a solution, can include but is not limited to one or more following anti virus drugs: remdesivir, Acyclovir, Valacyclovir, Ganciclovir, Valganciclovir, Foscarnet, Cidofovir, Amantadine, Rimantadine, Oseltamivir, Zanamivir, ribavirin, Adefovir, Emtricitabine, Entecavir, Lamivudine, Telbivudine, Tenofovir, Boceprevir, Telaprevir.

### 1.1 Liquid Phases

In the present disclosure, pharmaceutical formulations produced by reverse emulsification process can have the mixture of fluorinated and/or perfluorinated liquids compounds as the continuous phase. Pharmaceutical formulations produced by mixing the liquid perflurocarbon emulsion with corticosteroid or prostacyclin emulsion can have aqueous inorganic salt buffers as the continuous phase. In case of pharmaceutical formulations where micro or nano particles of corticosteroid or prostacyclin are suspended using fluorinnated surfactants, the mixture of fluorinated and/or perfluorinated liquid compounds work as the continuous phase. Pharmaceutical formulation where corticosteroid or prostacyclin is dissolved in fluorinated and/or perfluorinated liquids by using a cosolvent, the continuous phase is the mixture of fluorinated and/or perfluorinated liquids compounds containing the dissolved cosolvent.

Despite their lipophobicity, FCs can slowly partition into lipid bilayers and erythrocyte membranes. Although systematic cellular uptake studies with a structurally diverse group of FCs have not been performed to date, it is likely that their uptake into cell membranes increases with increasing lipophilicity. This hypothesis is indirectly supported by the observation that the elimination of FCs, which occurs primarily via the lung, is directly proportional to their lipophilicity and, thus, their uptake by cells in the lung. The lipophilicity of (nonfunctionalized) FCs is highly dependent on their molecular structure and decreases in the order of tricyclic>bicyclic>monocyclic>aliphatic; whereas the introduction of polarizable functional groups, such as bromine, increases the lipophilicity of FCs.

### 1.2 List of Exemplary Liquid Fluorocarbons

In the present disclosure the fluorinated and/or perfluorinated liquids used can include, but is not limited to, one or more liquidselected from: perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide). 1H,4H-perfluorobutane, 1H-PERFLUOROPENTANE, HFA 134a™, HFA227ea™, methyl perfluorobutylether, methyl perfluoropropyl ether (3M Novec 7000™), 2,2,2-trifluoroethanol and combinations thereof. These fluorinated and/or perfluorinated liquids cover boiling point ranges which both above and below the physiological temperature 37° C. In the current disclosure the fluorinated and/or perfluorinated liquids boils off near body temperature loosening the phlegm from the airways to facilitate the natural breathmthyl ing. The mixture of fluorinated and/or perfluorinated liquids can have boiling point greater than about 30° C., about 35° C., about 40° C., about 45° C., about 50° C. Table 4 provides a list of certain perfluorinated liquids and their respective boiling points.

TABLE 4
Liquid fluorocarbons and their boiling points

| Perfluorinated | Boiling |
|---|---|
| liquids | point ° C. |
| perfluoropropane | −36.7 |
| Perfluorobutane | −1.7 |
| perfluoropentane | 28.0 |
| Perfluorohexane | 56.0 |
| perfluoroheptane | 56.0 |
| Perfluorooctane | 103.0 |
| Perfluorodecalin | 142 |
| Perfluoroperhydrophenanthrene | 215 |
| Perfluorooctylbromide | 142 |
| perfluoro tributyl amine | 178 |
| perfluorotripentyl amine | 215 |

### 1.2.1 Cosolvents

Corticosteroids and prostacyclins are not soluble in fluorinated and/or perfluorinated liquids. A mixture of cosolvent is required to prepare a stable solution of corticosteroid or prostacyclin in fluorinated and/or perfluorinated liquids. In the current disclosure the cosolvents can include but not limited to one or more alcohols (,g. ethanol, propanol, isopropanol, butanol, isobutanol). The choice of surfactant is helps to stabilize the emulsion mixture where corticosteroid or prostacyclin emulsion is prepared separately and mixed with a pre-emulsified fluorinated and/or perfluorinated liquids. Electrostatic charge and polarity of corticosteroid or prostacyclin emulsion is the same as the electrostatic charge and polarity of fluorinated and/or perfluorinated liquids emulsion. Surfactant used for both emulsions can include but not limited to a mixture of phosphoglycolipid (DSPC, DSPE, DSPS etc), synthetically modified lipid polymer conjugate (DSPE-PEG)or amphiphilic polymer (e.g. polylactide-co-glycolide)or fluorinated polymeric surfactant (PEG-krytox). Surfactants used in the dispersion of corticosteroid/prostacyclin by reverse emulsion or in the nano/micro particle can include but not limited to liner polyether polymers containing distinct hydrogenated and perfluorinated blocks, hydrofluoroalkane (e.g. 1H,4H-perfluorobutane, 1H-perfluoropentane, HFA 134a™, HFA227ea™), hydrofluoroether (e.g.methyl perfluorobutylether, methyl perfluoropropyl ether (3M Novec 7000™)), hydrofluoro alcohols (e.g, 2,2,2-tritluoroethanol),

### 1.3 Surfactants

Natural glycophospholipids listed in table 5 are more suitable for the emulsification of corticosteroid or prostacyclin or fluorinated and/or perfluorinated liquids compounds. For the particle suspension hydrofluoroether, hydrofluoroalkane or hydrofluoroalcohol are preferred due to its volatility under physiological temperature.

TABLE 5
Lipid surfactants-abbreviations used and chemical information of
glycerophospholipids

| Abbreviation | CAS | Name | Type |
|---|---|---|---|
| DDPC | 3436-44-0 | 1,2-Didecanoy1-sn-glycero-3- | Phosphatidylcholine |
| | | phosphocholine | |
| DEPA-NA | 80724-31-8 | 1,2-Dierucoyl-sn-glycero-3- | Phosphatidic acid |
| | | phosphate (Sodium Salt) | |
| DEPC | 56649-39-9 | 1,2-Dierucoyl-sn-glycero-3- | Phosphatidylcholine |
| | | phosphocholine | |
| DEPE | 988-07-2 | 1,2-Dierucoyl-sn-glycero-3- | Phosphatidylethanolamine |
| | | phosphoethanolamine | |
| DEPG-NA | | 1,2-Dierucoyl-sn-glycero- | Phosphatidylglycerol |
| | | 3[Phospho-rac-(1-glycerol . . . ) | |
| | | (Sodium Salt) | |
| DLOPC | 998-06-1 | 1,2-Dilinoleoyl-sn-glycero-3- | Phosphatidylcholine |
| | | phosphocholine | |
| DLPA-NA | | 1,2-Dilauroyl-sn-glycero-3- | Phosphatidic acid |
| | | phosphate (Sodium Salt) | |
| DLPC | 18194-25-7 | 1,2-Dilauroyl-sn-glycero-3- | Phosphatidylcholine |
| | | phosphocholine | |
| DLPE | | 1,2-Dilauroyl-sn-glycero-3- | Phosphatidylethanolamine |
| | | phosphoethanolamine | |
| DLPG-NA | | 1,2-Dilauroyl-sn-glycero- | Phosphatidylglycerol |
| | | 3[Phospho-rac-(1-glycerol . . . ) | |
| | | (Sodium Salt) | |
| DLPG-NH4 | | 1,2-Dilauroyl-sn-glycero- | Phosphatidylglycerol |
| | | 3[Phospho-rac-(1-glycerol . . . ) | |
| | | (Ammonium Salt) | |
| DLPS-NA | | 1,2-Dilauroyl-sn-glycero-3- | Phosphatidylserine |
| | | phosphoserine (Sodium Salt) | |
| DMPA-NA | 80724-3 | 1,2-Dimyristoyl-sn-glycero-3- | Phosphatidic acid |
| | | phosphate (Sodium Salt) | |
| DMPC | 18194-24-6 | 1,2-Dimyristoyl-sn-glycero-3- | Phosphatidylcholine |
| | | phosphocholine | |
| DMPE | 988-07-2 | 1,2-Dimyristoy1-sn-glycero-3- | Phosphatidylethanolamine |
| | | phosphoethanolamine | |
| DMPG-NA | 67232-80-8 | 1,2-Dimyristoyl-sn-glycero- | Phosphatidylglycerol |
| | | 3[Phospho-rac-(1-glycerol . . . ) | |
| | | (Sodium Salt) | |
| DMPG-NH4 | | 1,2-Dimyristoy1-sn-glycero- | Phosphatidylglycerol |
| | | 3[Phospho-rac-(1-glycerol . . . ) | |
| | | (Ammonium Salt) | |
| DMPG- | | 1,2-Dimyristoyl-sn-glycero- | Phosphatidylglycerol |
| NH4/NA | | 3[Phospho-rac-(1-glycerol . . . ) | |
| | | (Sodium/Ammonium Salt) | |
| DMPS-NA | | 1,2-Dimyristoyl-sn-glycero-3- | Phosphatidylserine |
| | | phosphoserine (Sodium Salt) | |
| DOPA-NA | | 1,2-Dioleoyl-sn-glycero-3- | Phosphatidic acid |
| | | phosphate (Sodium Salt) | |
| DOPC | 4235-95-4 | 1,2-Dioleoyl-sn-glycero-3- | Phosphatidylcholine |
| | | phosphocholine | |
| DOPE | 4004-5-1- | 1,2-Dioleoyl-sn-glycero-3- | Phosphatidylethanolamine |
| | | phosphoethanolamine | |
| DOPG-NA | 62700-69-0 | 1,2-Dioleoyl-sn-glycero- | Phosphatidylglycerol |
| | | 3[Phospho-rac-(1-glycerol . . . ) | |
| | | (Sodium Salt) | |
| DOPS-NA | 70614-14-1 | 1,2-Dioleoyl-sn-glycero-3- | Phosphatidylserine |
| | | phosphoserine (Sodium Salt) | |
| DPPA-NA | 71065-87-7 | 1,2-Dipalmitoyl-sn-glycero-3- | Phosphatidic acid |
| | | phosphate (Sodium Salt) | |
| DPPC | 63-89-8 | 1,2-Dipalmitoyl-sn-glycero-3- | Phosphatidylcholine |
| | | phosphocholine | |
| DPPE | 923-61-5 | 1,2-Dipalmitoyl-sn-glycero-3- | Phosphatidylethanolamine |
| | | phosphoethanolamine | |
| DPPG-NA | 67232-81-9 | 1,2-Dipalmitoyl-sn-glycero- | Phosphatidylglycerol |
| | | 3[Phospho-rac-(1-glycerol . . . ) | |
| | | (Sodium Salt) | |
| DPPG-NH4 | 73548-70-6 | 1,2-Dipalmitoyl-sn-glycero- | Phosphatidylglycerol |
| | | 3[Phospho-rac-(1-glycerol . . . ) | |
| | | (Ammonium Salt) | |
| DPPS-NA | | 1,2-Dipalmitoyl-sn-glycero-3- | Phosphatidylserine |
| | | phosphoserine (Sodium Salt) | |
| DSPA-NA | 108321-18-2 | 1,2-Distearoyl-sn-glycero-3- | Phosphatidic acid |
| | | phosphate (Sodium Salt) | |
| DSPC | 816-94-4 | 1,2-Distearoyl-sn-glycero-3- | Phosphatidylcholine |
| | | phosphocholine | |
| DSPE | 1069-79-0 | 1,2-Distearoyl-sn-glycero-3- | Phosphatidylethanolamine |
| | | phosphoethanolamine | |
| DSPG-NA | 67232-82-0 | 1,2-Distearoyl-sn-glycero- | Phosphatidylglycerol |
| | | 3[Phospho-rac-(1-glycerol . . . ) | |
| | | (Sodium Salt) | |
| DSPG-NH4 | 108347-80-4 | 1,2-Distearoyl-sn-glycero- | Phosphatidylglycerol |
| | | 3[Phospho-rac-(1-glycerol . . . ) | |
| | | (Ammonium Salt) | |
| DSPS-NA | | 1,2-Distearoyl-sn-glycero-3- | Phosphatidylserine |
| | | phosphoserine (Sodium Salt) | |
| EPC | | Egg-PC | Phosphatidylcholine |
| HEPC | | Hydrogenated Egg PC | Phosphatidylcholine |
| HSPC | | Hydrogenated Soy PC | Phosphatidylcholine |
| LYSOPC | 18194-24-6 | 1-Myristoyl-sn-glycero-3- | Lysophosphatidylcholine |
| MYRISTIC | | phosphocholine | |
| LYSOPC | 17364-16-8 | 1-Palmitoyl-sn-glycero-3- | Lysophosphatidylcholine |
| PALMITIC | | phosphocholine | |
| LYSOPC | 19420-57-6 | 1-Stearoyl-sn-glycero-3- | Lysophosphatidylcholine |
| STEARIC | | phosphocholine | |
| Milk | | 1-Myristoyl-2-palmitoyl-sn- | Phosphatidylcholine |
| Sphingomyelin | | glycero 3-phosphocholine | |
| MPPC | | | |
| MSPC | | 1-Myristoyl-2-stearoyl-sn- | Phosphatidylcholine |
| | | glycero-3-phosphocholine | |
| PMPC | | 1-Palmitoyl-2-myristoyl-sn- | Phosphatidylcholine |
| | | glycero-3-phosphocholine | |
| POPC | 26853-31-6 | 1-Palmitoyl-2-oleoyl-sn- | Phosphatidylcholine |
| | | glycero-3-phosphocholine | |
| POPE | | 1-Palmitoyl-2-oleoyl-sn- | Phosphatidylethanolamine |
| | | glycero-3- | |
| | | phosphoethanolamine | |
| POPG-NA | 81490-05-3 | 1-Palmitoyl-2-oleoyl-sn- | Phosphatidylglycerol |
| | | glycero-3[Phospho-rac-(1- | |
| | | glycerol) . . . ] (Sodium Salt) | |
| PSPC | | 1-Palmitoyl-2-stearoyl-sn- | Phosphatidylcholine |
| | | glycero-3-phosphocholine | |
| SMPC | | 1-Stearoyl-2-myristoyl-sn- | Phosphatidylcholine |
| | | glycero-3-phosphocholine | |
| SOPC | | 1-Stearoyl-2-oleoyl-sn- | Phosphatidylcholine |
| | | glycero-3-phosphocholine | |
| SPPC | | 1-Stearoyl-2-palmitoyl-Sn- | Phosphatidylcholine |
| | | glycero-3-phosphocholine | |

### 2.0 Method of Preparing Cortcosteroid/Prostacyclin/Antibiotic/Antiviral Drug Pharmaceutical Compositions

### 2.1 Methods of Preparing corticosteroid/prostacyclin/Antibiotic/Anti Virus Drug Dispersions by Reverse Emulsification

Corticosteroid/prostacyclin/antibiotic/anti virus drug and fluorinated surfactant are dissolved in ethanol then the resulting solution is slowly added to the mixture of fluorinated and/or perfluorinated liquids and sonicated to disperse uniformly. The concentration of corticosteroid ranges from about 5-10 mcg/mL, about 10-15, mcg/mL, about 15-20 mcg/mL, about 20-25 mcg/mL, about 25-30 mcg/mL, about 30-35 mcg/mL, about 35-40 mcg/mL, about mcg/mL or about 45-50 mcg/mL.

### 2.2 Methods of Preparing the corticosteroid/prostacyclin and Fluorinated and/or Perfluorinated Liquids Emulsion Mixture

In a typical procedure corticosteroid or prostacyclin is first dissolved in ethanol then the resulting solution is slowly added to a saline solution containing DSPC surfactant. Solution was sonicated to uniformly disperse the ethanolic solution of corticosteroid or prostacyclin. Similarly, the fluorinated and/or perfluorinated liquids emulsion is prepared by dispersing the fluorinated and/or perfluorinated liquids in saline solution containing phospholipid surfactant. Finally, both emulsions are mixed to produce the emulsion mixture.
The concentration of corticosteroid ranges from about 5-10 mcg/mL, about 10-15 mcg/mL, about 15-20 mcg/mL, about 20-25 mcg/mL, about 25-30 mcg/mL, about 30-35 mcg/mL, about 35-40 mcg/mL, about 40-45 mcg/mL or about 45-50 mcg/mL.

### 2.3 Methods of Preparing Particle Suspensions

2.3.1 Preparation of corticosteroid Micro/Nano Particles

Corticosteroid/prostacyclin nano/microparticles are prepared by electrospraying or ball milling process. A typical electrospray process involves dissolving corticosteroid/prostacyclin in ethanol or trifluoroethanol containing a fluorinated surfactant, followed by electrospray under high voltage. Particle size varies with the voltage and fluorinated surfactant concentration. Resulting micro/nano particles are then suspended in a mixture of fluorinated and/or perfluorinated liquids, fluorinated surfactant and cosolvent.

Corticosteroid/prostacyclin nano/micro particles are also prepared by nano milling process. Particle suspension is prepared by dispersing the nanolmicroparticl es into a mixture of fluorinated and/or perfluorinated liquids fluorinated surfactant and cosolvent. The nano/micro particle suspension of corticosteroid/prostacyclin is also directly prepared by dispersing solid corticosteroid/prostacyclin under high shear into the mixture of fluorinated and/or perfluorinated liquids, fluorinated surfactant and cosolvent.

### 2.3.2 Particle Size Determination

Particle size in the suspension is determined by dynamic light scattering technique. Dynamic light scattering technique is also used for determining the particle size distribution of the corticosteroid/prostacyclin emulsion and fluorinated and/or perfluorinated liquids emulsion and the mixture of both emulsions.

### 2.3.3 Compositions Comprising Particles

Dispersions of corticosteroid/prostacyclin nano/microparticle in the mixture of fluorinated and/or perfluorinated liquids comprise of corticosteroid/prostacyclin concentration ranging from about 5-10 mcg/mL, about 10-15 mcg/mL, about 15-20 mcg/mL, about 20-25 mcg/mL, about 25-30 mcg/mL, about 30-35 mcg/mL, about 35-40 mcg/mL, about 40-45 mcg/mL or about 45-50 mcg/mL.

Dispersion of corticosteroid/prostacyclin nano/microparticle in the mixture of fluorinated and/or perfluorinated liquids comprises of cosolvents concentration less than about 1.0%, about 2%, about 5%, about 10%, about 15%, about 20%.

Dispersion of corticosteroid/prostacyclin nano/microparticle in the mixture of fluorinated and/or perfluorinated liquids comprises of fluorinated surfactant concentration ranging from about 0.5-1%, about 1-2%, about 2-3%, about 3-4%, about 4-5%, about 5-6%, about 6-7%, about 7-8%, about 8-9% or about 9-10% on a weight basis.

### 2.4 Methods of Preparing Solutions

In a typical procedure for preparing the corticosteroid/prostacyclin solution in the mixture of fluorinated and/or perfluorinated liquids the corticosteroid/prostacyclin is dissolved in a cosolvent mixture first then the resulting solution is diluted in a mixture of fluorinated and/or perfluorinated liquids compounds.

Solution of corticosteroid/prostacyclin in the mixture of fluorinated and/or perfluorinated liquids comprises of corticosteroid/prostacyclin concentration ranging from 5-10 mcg/mL, about 10-15 mcg/mL, about 15-20 mcg/mL, about 20-25 mcg/mL about 25-30 mcg/mL, about 30-35 mcg/mL, about 35-40 mcg/mL, about 40-45 mcg/mL or about 45-50 mcg/mL.

Solution of corticosteroid/prostacyclin nanolmicroparticle in the mixture of fluorinated and/or perfluorinated liquids comprises of cosolvents concentration less than about 1.0%, about 2%, about 5%, about 10%, about 15%, about 20%.

### 3.0 Methods of Administration and Treatment

### 3.1 Method of Treatment

The present disclosure features the treatment of subject suffering from pneumonia, bronchiolitis, asthma or COPD. In one aspect the present disclosure relates to a treatment of mammals, especially humans, suffering from viral pneumonia. In another aspect the present disclosure relates to a treatment of a mammals, especially humans suffering from bacterial pneumonia. In another aspect the present disclosure relates to a treatment of mammals, especially humans suffering from asthma or COPD. Again in another aspect the present disclosure relates to a precautionary treatment of mammals, especially humans suffering from chronic asthma or COPD and exposed to a viral pandemic or community infection which is defined as the spread of infections via social interactions.

### 3.1.1 Treatment

Pharmaceutical formulations comprising of corticosteroid in fluorinated and/or perfluorinated liquids will be delivered through nebulizer to treat early or late stage viral pneumonia. Pharmaceutical formulations comprising of antibiotic and liquid perfluorcarbon will also be nebulized to treat bacterial pneumonia after clinical diagnosis. Pharmaceutical formulations comprising of prostacyclin or nitric oxide or albuterol and fluorinated and/or perfluorinated liquids will also be nebulized to manage asthma or COPD attack.

### 3.1.2 Dosage and Frequency

Pharmaceutical formulations mixture comprising of corticosteroid in fluorinated and/or perfluorinated liquids will be nebulized and inhaled through an introduction assembly that includes a face mask or ventilator. Single dose can contain about 1 mL, about 2 mL, about 3 mL, about 4 mL or about 5 mL of the foi nulation mixture. Formulation will be delivered daily once twice or three times depending on the severity of hypoxia.

### EXAMPLES

### Example 1

System to oxygenate ARDS subjects: ARDS subject breathes in an aerosolized mixture of fluorinated and /perfluorinated liquid containing 80% perfluoro pentane and 20% perfluorodecalin at 0.8 mL/min rate and optionally exhales perfluoro pentane resulting in evaporative cooling at 0.5° C./min rate in the pulmonary tissue whereas the higher boiling perfluorodecalin will collect in alveoli and deliver the dissolved oxygen to alveolar tissue.

Exhaled fluorinated and /perfluorinated liquid is captured, oxygenated and aerosolized back to pulmonary tissue along with a metered dose of moisture. Perfluorodecalin also completely evaporates and is exhaled from the pulmonary tissue over the next 24 hours.

### Example 2

System to deliver active pharmaceutical ingredients to ARDS subject pulmonary tissue to reduce inflammation: ARDS subject breaths in an aerosolized mixture of fluorinated and /perfluorinated liquid containing 69.9% perfluoropetrtane and 20% perfluorodecalin formulated with 10% alcohol (such as ethanol, for example) and 0.1% corticosteroid at 10 mL/min rate and optionally exhales perfluoro pentane resulting in evaporative cooling at 1° C./min rate in the lung to reduce inflammation. Exhaled fluorinated and /perfluorinated liquid is captured, oxygenated and aerosolized back to pulmonary tissue along with a metered and/or controlled dose of moisture.

### Example 3

System for lung oxygenation and cooling for asthma subject: Asthma or COPD subject breaths in an aerosolized mixture of fluorinated and /perfluorinated liquid containing 68.9% perfluoro pentane and 20% perfluorodecalin foimulated with 10% alcohol (such as ethanol, for example), 1% surfactant and 0.1% albuterol at 10 ml/min rate and optionally exhales perfluoro pentane resulting in evaporative cooling at 1° C./min rate in the lung to reduce inflammation. Exhaled fluorinated and /perfluorinated liquid is captured, oxygenated and aerosolized back to pulmonary tissue along with a metered and/or controlled dose of moisture.

### Example 4

System for efficient lung recruitment and pulmonary delivery of corticosteroid to viral pneumonia subject: Viral pneumonia subject breaths in an aerosolized mixture of fluorinated and /perfluorinated liquid containing 68.9% perfluoro pentane and 20% perfluorodecalin formulated with 10% alcohol (such as ethanol, for example), 1% surfactant and 0.1% corticosteroid at 10 mL/min rate and optionally exhales perfluoro pentane resulting in evaporative cooling at 1° C./min rate in the lung to reduce inflammation. Exhaled fluorinated and /perfluorinated liquid is captured, oxygenated and aerosolized back to pulmonary tissue along with a metered and/or controlled dose of moisture.

### Example 5

System for efficient lung recruitment and pulmonary delivery of corticosteroid to bacterial pneumonia subject: Bacterial pneumonia subject breaths in an aerosolized mixture of fluorinated and /perfluorinated liquid containing 68.9% perfluoro pentane and 20% perfluorodecalin formulated with 10% alcohol (such as ethanol, for example), 1% surfactant and 0.1% antibiotic at 10 mL/rain rate and optionally exhales perfluoro pentane resulting in evaporative cooling at 1° C./min rate in the lung to reduce inflammation. Exhaled fluorinated and /perfluorinated liquid is captured, oxygenated and aerosolized back to pulmonary tissue along with a metered dose of moisture.

### Example 6

System for lung oxygenation and cooling for asthma subject: Inhalation hazard induced acute lung injury subject receives an aerosolized lavage mixture of fluorinated and /perfluorinated liquid containing 80% perfluorodecalin and 20% perfluoro pentane at 50 mL/min rate and optionally exhales perfluoro pentane resulting in evaporative coiling at 1° C./rain rate in the pulmonary tissue whereas the higher boiling perfluorodecalin gets collected in alveoli and deliver the dissolved oxygen to alveolar tissue at maximum 25 mL/min rate. Collected perfluorodecalin is exhaled or mechanically suctioned.

### Example 7

System or lung oxygenation and cooling for asthma subject: Inhalation hazard induced acute lung injury subject receives an aerosolized lavage of saline based fluorinated and /perfluorinated liquid emulsion containing 40% perfluorodecalin, 10% perfluoro pentane and 49% saline with 1% dissolved therapeutics at 50 ml/min rate and optionally exhales perfluoro pentane resulting in evaporative cooling at 1° C./min rate in the pulmonary tissue whereas the higher boiling perfluorodecalin gets collected in alveoli and deliver the dissolved oxygen to alveolar tissue at maximum 25 mL/min rate. Residual saline and fluorinated and /perfluorinated liquids are exhaled or mechanically suctioned at the end of the lavage cycle.

### Example 8

System for deceased subject's lung cooling to preserve the organ during transplant: An aerosolized mixture of fluorinated and /perfluorinated liquid containing 80% perfluoro pentane and 20% perfluorodecalin at 10 mL/min rate and optionally exhales perfluoro pentane resulting in evaporative cooling at 1° C./min rate in the pulmonary tissue whereas the higher boiling perfluorodecalin gets collected in alveoli and delivers the dissolved oxygen to alveolar tissue at a maximum 5 mL/min rate. Exhaled fluorinated and /perfluorinated liquid is captured, oxygenated and aerosolized back to pulmonary tissue along with a metered dose of moisture.

Disclosed herein is an apparatus for the treatment of lung tissue, the apparatus comprising: an inlet or reservoir for containing a one or more liquids to be aerosolized; a gas delivery regulator for controlling the flow of a delivery gas; an aerosolizer in fluid communication with the inlet or reservoir, and in gas flow communication with the gas delivery regulator for producing an aerosol of the liquid to be aerosolized in the delivery gas as an aerosol in the delivery gas; a feed tube, which is in gas flow communication with the aerosolizer, wherein the feed tube directs the aerosol in the delivery gas to the pulmonary system of a mammal or mammalian cadaver causing contact between the aerosol and pulmonary tissue, and wherein the aerosol is fully or partially volatilized in the delivery gas to form a gas that has been contacted with the pulmonary system; and a collection tube in gas flow communication with a condensation system. The collection tube directs gases that have been in contact with the pulmonary system to a condensation system. The condensation system recovers all or part of at least one component of the liquids to be aerosolized from gases that have been contacted with the pulmonary system as a condensed liquid. The apparatus further includes a separation system in fluid communication with the condensation system that separates all or some of the water from the at least one component of the liquids to be aerosolized present in the condensed liquid; and a return system is in fluid communication with the separation system and the reservoir and/or the aerosolizer, wherein the return system receives the at least one component of the liquids to be aerosolized and facilitates flow of all or part of the at least one component of the liquid to be aerosolized to the reservoir and/or aerosolizer.

Disclosed herein is an apparatus that recirculates a mixture comprising at least one fluorinated hydrocarbon, molecule with a fluorinated hydrocarbon radical, molecule with a perfluorinated hydrocarbon radical, or perfluorinated hydrocarbon (FC) to between that apparatus and the pulmonary tissue of a mammal or mammalian cadaver.

Disclosed herein is an apparatus for the treatment of lung tissue comprising: an inlet or reservoir for containing a one or more liquids to be aerosolized; a gas delivery regulator for controlling the flow of a delivery gas; an aerosolizer in fluid communication with the inlet or reservoir, and in gas flow communication with the gas delivery regulator for producing an aerosol of the liquid to be aerosolized in the delivery gas as an aerosol in the delivery gas; and a feed tube, which is in gas flow communication with the aerosolizer, wherein the feed tube directs the aerosol in the delivery gas to the pulmonary system of a mammal or mammalian cadaver causing contact between the aerosol and pulmonary tissue, and wherein the aerosol is fully or partially volatilized in the delivery gas to form a gas that has been contacted with the pulmonary system. In some embodiments, the apparatus further includes: a collection tube in gas flow communication with a condensation system; wherein the collection tube directs gases that have been in contact with the pulmonary system to a condensation system, (wherein the condensation system recovers all or part of at least one component of the liquids to be aerosolized from gases that have been contacted with the pulmonary system as a condensed liquid); a separation system in fluid communication with the condensation system that separates all or some of the water from the at least one component of the liquids to be aerosolized present in the condensed liquid; and a return system in fluid communication with the separation system and the reservoir and/or the aerosolizer, wherein the return system receives the at least one component of the liquids to be aerosolized and facilitates flow of all or part of the at least one component of the liquid to be aerosolized to the reservoir and/or aerosolizer.

In some embodiments, the components of the liquids to be aerosolized are delivered to the inlet or reservoir from separate supplies external to the apparatus. In some embodiments, the components of the liquids to be aerosolized are delivered to the inlet or reservoir from containers that are part of the apparatus. In some embodiments, the inlet or reservoir comprises a mixer for combining the liquids to be aerosolized. In some embodiments, the inlet or reservoir comprises temperature regulator, chiller, or warmer for heating and/or cooling the at least one of the liquids to be aerosolized. In some embodiments, the aerosolizer is a jet, forced air, ultrasonic; or piezoelectric device. In some embodiments, the gas delivery regulator controls the flow, pressure, or both the flow and pressure of the delivery gas.

In some embodiments, the condensation system cools the gases that have been in contact with the pulmonary system to cause condensation of all or part of at least one component of the liquids to be aerosolized from gases that have been contacted with the pulmonary system. The condensation system can cool and either raise or reduce the pressure of the gases that have been contacted with the pulmonary system in the process of causing condensation of all or part of at least one component of the liquids to be aerosolized from gases that have been contacted with the pulmonary system.

In some embodiments, the separation system separates at least 80% of the water from the from the at least one component of the liquids to be aerosolized present in the condensed liquid. For example, the separation system can separate from about 80% to about 90%, from about 90% to about 95%, or from about 95% to 100% of the water from the at least one component of the liquids to be aerosolized present in the condensed liquid. The return system can include a filter for removing any particulate material from the at least one component of the liquids to be aerosolized prior to the reservoir and/or aerosolizer. The filter can have a size cutoff of 0.2 microns or less (e.g., 0.1, or 0.05 microns or less).

In some embodiments, the feed tube is attached to a mechanical ventilator, and the aerosol in the delivery gas is delivered to the pulmonary system through either a the mechanical ventilator; a forced air system (the aerosol in the delivery gas is delivered to the pulmonary system through the forced air system through the mouth or nose); or a mask covering the nose and/or mouth, and the aerosol in the delivery gas is delivered to the pulmonary system through the mask by spontaneous breathing. The collection tube can be connected to the mechanical ventilator, forced air system or mask. The feed tube and/or collection tube can be outfitted with at least one valve that prevents or substantially prevents: (i) gases that have been contacted with the pulmonary system from flowing from the mammal or cadaver toward the aerosolizer via the feed tube; and/or (ii) gases that have been contacted with the pulmonary system from flowing from the condensation system to the mammal or cadaver via the collection tube. Some embodiments include a suction catheter for insertion into the pulmonary system. The suction catheter draws liquid from the pulmonary system or the pulmonary tissue.

In some embodiments, the delivery gas comprises air, oxygen; nitrogen; heliox, or a mixture thereof. Some embodiments include a temperature controller for warming or cooling the aerosol in the delivery gas prior to contacting it with the pulmonary tissue of the mammal or cadaver.

In some embodiments, the droplets of aerosol in the delivery gas have a mean diameter less than 2 microns. In some embodiments, the droplets of aerosol in the delivery gas has a mean diameter in a range selected from about 0.05 to about 0.1 microns, from about 0.1 to about microns, from about 0.4 to about 0.8 microns, from about 0.8 to about 1.2 microns, or from about 1.2 to about 2.0 microns, from about 2.0 microns to 5.0 microns. In some embodiments, the apparatus can deliver up to 10, 20, 30, 40, 50, or 60 ml/minute of the liquids to be aerosolized in aerosol form to the pulmonary system of the subject.

In some embodiments, the liquid to be aerosolized comprises at ast one liquid with a boiling point from about −2° C. to about 300° C. degrees C. at sea level. In some embodiments, the liquid to be aerosolized comprises at least one liquid with a boiling point from about 25° C. to about 150° C. at sea level. In some embodiments, the liquid to be aerosolized comprises at least one liquid with a boiling point from about 30° C. to about 140° C. at sea level. In some embodiments, the liquid to be aerosolized comprises at least on liquid with boiling point at sea level greater than about 36° C. In some embodiments, the liquid aerosolized in the delivery gas is saturated or partially saturated with the delivery gas.

In some embodiments, the liquid to be aerosolized comprises at least a fluorinated or a perfluorinated molecule. In some embodiments, the liquid to be aerosolized comprises at least one fluorinated hydrocarbon, molecule with a fluorinated hydrocarbon radical, molecule with a perfluorinated hydrocarbon radical, or perfluorinated hydrocarbon (FC). In some embodiments, the liquid to be aerosolized comprises at least one fluorocarbon or fluorocarbon, or a combination of at least one fluorocarbon and at least one fluorocarbon, or a combination of at least two fluorocarbons. In some embodiments, the liquid to be aerosolized comprises: the fluorinated and/or perfluorinated liquids which can include, but is not limited to, one or more selected from: perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), hydrofluoroalkane (e.g. 1H,4H-perfluorobutane, lh-perfluoropentane, HFA 134a™, HFA227ea™), hydrofluoroether (e.g.methyl perfluorobutylether, methyl perfluoropropyl ether (3M Novec 7000™)), hydrofluoro alcohols (e.g. 2,2,2-trifluoroethanol) and combinations thereof.

In some embodiments, the liquid to be aerosolized further comprises an active pharmaceutical ingredient (API). In some embodiments, the active pharmaceutical ingredient comprises a corticosteroid, prostacyclin, antibiotic, and/or an anti-virus drug.

Disclosed herein are pharmaceutical compositions for delivery to the pulmonary system of a subject. The pharmaceutical compositions can include one or more corticosteroids/prostacyclin/antibiotics/anti-virus drugs, a mixture of fluorinated and/or perfluorinated liquids, a cosolvent and a fluorinated surfactant, wherein at least one corticosteroid/prostacyclin/antibiotic/anti-virus drug is dispersed in the form of a reverse emulsion in the continuous phase of fluorinated and/or perfluorinated liquid. In some embodiments, the corticosteroid/prostacyclin/antibiotic/anti-virus drug is dispersed in a mixture of fluorinated and/or perfluorinated liquids in the form of a reverse emulsion, the corticosteroid/prostacyclin/antibiotic/anti virus drug is first dissolved in a hydrophilic cosolvent e.g. alcohol or water before dispersing it in the form of reverse emulsion.

Some embodiments of the pharmaceutical compositions include an optional concentration of dissolved oxygen in the continuous phase fluorinated and/or perfluorinated liquid. The concentration of oxygen ranges from 0-5%, 5-10%, 10-15%, 15-20%, 20-25%, 25-30%, 30-35%, 35-40%, 40-45%, 45-50% by volume.

In some embodiments, the solution of corticosteroid/prostacyclin/antibiotic/anti-virus drug is dispersed in the mixture of fluorinated and/or perfluorinated liquids, the particle size of the dispersed droplets ranges from 5-10 nm, 10-30 nm, 30-100 nm, 100-300 nm, 300-500 nm, 500-750 nm, 750 nm-1 μm, 1-10 μm as determined by dynamic light scattering technique.

Disclosed herein are pharmaceutical compositions including a preformed emulsion of corticosteroid/prostacyclin/antivirus drug and another preformed emulsion of fluorinated and/or perfluorinated liquid, wherein both emulsion droplets are coalesced together or independently dispersed in the continuous aqueous phase containing dissolved inorganic salts. In some embodiments, two preformed emulsions are mixed together, the electrostatic charge of the surfactant used for the individual emulsions are either both cationic, anionic or neutral at pH 7.4. In some embodiments, two prefoinied emulsions are mixed together the particle size of the emulsion mixture ranges from 5-10 nm, 10-30 nm, 30-100 nm, 100-300 nm, 300-500 nm, 500-750 nm, 750 nm-1 μm, 1-10 μm as determined by dynamic light scattering technique. In some embodiments, two preformed emulsions are mixed together the concentration of dissolved oxygen in the preformed emulsion of fluorinated and/or perfluorinated liquid ranges from 0-5%, 10-15%, 15-20%, 20-25%, 25-30%, 30-35%, 35-40%, 40-45%, 45-50% by volume.

Some embodiments include an optional concentration of dissolved oxygen in the emulsified fluorinated and/or perfluorinated liquid phase. The concentration of oxygen ranges from 0-5%, 10-15%, 15-20%, 20-25%, 25-30%, 30-35%, 35-40%, 40-45%, 45-50% by volume. In some embodiments, the corticosteroidlprostacyclinlantibiotic/anti-virus drug is dispersed by reverse emulsification process the corticosteroid/prostacyclin/antibiotic/anti-virus drug and fluorinated surfactant are dissolved in ethanol then the resulting solution is slowly added to the mixture of fluorinated and/or perfluorinated liquids and sonicated to disperse uniformly.

Disclosed herein are pharmaceutical compositions for delivery to the pulmonary system of a subject including one or more vasodilator or antibiotic or antivirus drug, a mixture of fluorinated and/or perfluorinated liquids, a cosolvent and a surfactant, wherein at least one vasodilator or antibiotic or antivirus drug is dissolved in the continuous aqueous phase with the help of an optional co-solvent and the mixture of fluorinated and/or perfluorinated liquids is emulsified using a surfactant. In some embodiments, vasodilator or antibiotic or antivirus drug is in the solution phase the concentration of antibiotic or anti-viral drug in the final composition ranges from 5-10 mg/mL, 10-20 mg/mL, 20-30 mg/mL, 30-40 mg/mL, 40-50 mg/mL, 50-100 mg/mL. In some embodiments, vasodilator or antibiotic or antivirus drug is in the solution phase the concentration of vasodilator in the final composition ranges from 0.1-0.5 mg/mL, 0.5-1.0 mg/mL, 1.0-1.5 mg/mL, 1.5-2.0 mg/mL. In some embodiments, vasodilator or antibiotic or antivirus drug is in the solution phase and the mixture of fluorinated and/or perfluorinated liquids is emulsified in that solution using a surfactant, the particle size of the emulsion ranges between 5-10 nm, 10-30 nm, 30-100 nm, 100-300 nm, 300-500 nm, 500-750 nm, 750 nm-1 μm, 1-10 μm as determined by dynamic light scattering technique. Some embodiments include an optional concentration of dissolved oxygen in the emulsified fluorinated and/or perfluorinated liquid phase. The concentration of oxygen ranges from 0-5%, 5-10%, 10-15%, 15-20%, 20-25%, 25-30%, 30-35%, 35-40%, 40-45%, 45-50% by volume. In some embodiments, where corticosteroid or prostacyclin emulsion is mixed with a liquid fluorocarbon/fluorocarbon emulsion, the corticosteroid or prostacyclin can be first dissolved in ethanol then the resulting solution is slowly added to aqueous solution of inorganic salts and phospholipid surfactant. Solution is sonicated to uniformly disperse the ethanolic solution of corticosteroid or prostacyclin. Similarly, the fluorinated and/or perfluorinated liquids emulsion can be prepared by dispersing the fluorinated and/or perfluorinated liquids in aqueous solution of inorganic salts and phospholipid surfactant. Finally, both emulsions can be mixed to produce the emulsion mixture.

Disclosed herein are pharmaceutical compositions for delivery to the pulmonary system of a subject including one or more corticosteroids/prostacyclin/antibiotics/anti-virus drugs, a mixture of fluorinated and/or perfluorinated liquids, a cosolvent and a fluorinated surfactant, wherein at least one corticosteroid/prostacyclin/antibiotic/anti virus drug is dispersed in the form of a nano/micro particles in the continuous phase of fluorinated and/or perfluorinated liquid. In some embodiments, the vasodilator or antibiotic or antivirus drug is dissolved in continuous aqueous phase, the vasodilator or antibiotic or antivirus drug is first dissolved in an alcohol then diluted in the aqueous solution of inorganic salts and phospholipid surfactants. Finally the mixture of fluorinated and/or perfluorinated liquids is emulsified in that solution by sonication.

In some embodiments, micro/nano particles of corticosteroids/prostacyclin/antibiotics/anti-virus drug is dispersed in the mixture of fluorinated and/or perfluorinated liquids, the micro/nano particles of corticosteroids/prostacyclin/antibiotics/anti-virus drug is produced by either by ball milling or by electrospraying or by nano milling or by a combination of electrospinning and ball milling process. In some embodiments, the micro/nano particles of corticosteroids/prostacyclin/antibiotics/anti virus drug can be dispersed in the mixture of fluorinated and/or perfluorinated liquids containing a fluorinated surfactant. In some embodiments the microlnano particle dispersion of corticosteroids/prostacyclin/antibiotics/anti virus drug in the pharmaceutical composition can be produced by dispersing lyophilized powder of corticosteroids/prostacyclin/antibiotics/anti virus drug under high shear in the mixture of fluorinated and/or perfluorinated liquids containing fluorinated surfactant. The lyophilized powder of corticosteroids/prostacyclin/antibiotics/anti-virus drug used for producing the pharmaceutical composition can be mixed with fluorinated surfactant before lyophilization and hence, can be dispersed into the mixture of fluorinated and/or perfluorinated liquids

Disclosed herein are pharmaceutical compositions for delivery to the pulmonary system of a subject including one or more corticosteroids/prostacyclin/antibiotics/anti-virus drugs, a mixture of fluorinated and/or perfluorinated liquids, a cosolvent and a fluorinated surfactant, wherein the composition is stable for 6 months at 20° C. or for 7 days at 37° C. when <5% change in particle size is observed by dynamic light scattering technique. Some embodiments include an optional concentration of dissolved oxygen in the continuous phase fluorinated and/or perfluorinated liquid. The concentration of oxygen ranges from 0-5%, 5-10%, 10-15%, 15-20%, 25-30%, 30-35%, 35-40%, 40-45%, 45-50% by volume.

Disclosed herein are pharmaceutical compositions for delivery to the pulmonary system of a subject including one or more corticosteroids/prostacyclin/anti-virus drugs, a mixture of fluorinated and/or perfluorinated liquids, a cosolvent wherein at least one corticosteroid/prostacyclin/anti-virus drug is dissolved in the continuous phase of fluorinated and/or perfluorinated liquid and cosolvent. In some embodiments, the corticosteroid/prostacyclin/anti-virus drug is dissolved in a mixture of fluorinated and/or perfluorinated liquids and a cosolvent the concentration of antivirus drug in the final composition ranges from 5-10 mg/mL, 10-20 mg/mL, 20-30 ing/mL, 30-40 mg/mL, 40-50 mg/mL, 50-100 mg/mL. In some embodiments, the corticosteroid/prostacyclin/anti-virus drug is dissolved in a mixture of fluorinated and/or perfluorinated liquids and a cosolvent the concentration of corticosteroid/prostacyclin in the final composition ranges from 5-10 mcg/mL, mcg/mL, 15-20 mcg/mL, 20-25 mcg/mL. 25-30 mcgimL, 30-35 mcg/mL, 35-40 mcg/mL, 40-45 mcg/mL or 45-50 mcg/mL. In some embodiments, the pharmaceutical composition can be produced by dissolving corticosteroid/prostacyclin/anti-virus drug in a hydrophilic solvent followed by diluting the solution with a mixture of fluorinated and/or perfluorinated liquids.

Some embodiments include a method of preparing the corticosteroid/prostacyclin solution in the mixture of fluorinated and/or perfluorinated liquids the corticosteroid/prostacyclin is dissolved in a cosolvent mixture first then the resulting solution is diluted in a mixture of fluorinated and/or perfluorinated liquids compounds.

Disclosed herein are pharmaceutical compositions for delivery to the pulmonary system of a subject including one or more corticosteroids/prostacyclin/anti-virus drugs, a mixture of fluorinated and/or perfluorinated liquids and a cosolvent wherein the composition is stable for 6 months at 20° C. when any solid separating out of the solution dissolves back to the solution phase as soon as the solution temperature reached 37° C. Some embodiments include an optional concentration of dissolved oxygen in the continuous phase fluorinated and/or perfluorinated liquid. The concentration of oxygen ranges from 0-5%, 5-10%, 10-15%, 15-20%, 25-30%, 30-35%, 35-40%, 40-45%, 45-50% by volume.

Disclosed herein is a method of cooling the pulmonary tissue and/or lung tissue of a mammal or a mammalian cadaver can include: forming and supplying the aerosol in the delivery gas by aerosolization of the liquids to be aerosolized in an apparatus as described above, and contacting the aerosol in the delivery gas with the pulmonary tissue and/or lungs of a mammal or a mammalian cadaver. The aerosol in the delivery gas can be cooled prior to contacting with the pulmonary tissue and/or lungs.

Disclosed herein is a method of exchanging gases within blood and/or tissues comprising: forming and supplying the aerosol in the delivery gas by aerosolization of the liquids to be aerosolized in an apparatus as described above, and contacting the aerosol in the delivery gas with the pulmonary tissue and/or lungs of a mammal or a mammalian cadaver.

Disclosed herein is a method of exchanging gases with blood in alveoli in pulmonary tissue by aerosolizing fluorocarbons, saturating them with desired gases, and delivering the aerosolized droplets to the alveoli.

Disclosed herein is a method of active pharmaceutical ingredient (API) delivery to the pulmonary tissue by mixing the API composition with the liquid to be aerosolized.

Disclosed herein is a method of cooling lung tissue which includes aerosolizing chilled fluids and delivering them to the pulmonary tissue. In some embodiments, the liquids to aerosolized are fluorocarbons and or fluorocarbons. In some embodiments, the boiling points of some or all of the liquids to be aerosolized are near or below 37C. In some embodiments, the method of cooling is applied to a deceased person to lower their lung temperature to preserve lung tissues and other body organs for organ transplant. In some embodiments, some amount of oxygen gas is mixed with the liquid to be aerosolized either before or after it is aerosolized to provide oxygen to the lung tissue.

Disclosed herein is a method of removing pus, water, mucus, and other contaminants from pulmonary tissue which includes aerosolizing a liquid and delivering it to the pulmonary tissue to coat the lungs and fill some portion of the alveoli (i.e., lung lavage). In some embodiments, the liquid to be aerosolized is one or more fluorocarbons and or fluorocarbons. In some embodiments, up to 200 ml of the liquid to be aerosolized accumulates in the lungs. In some embodiments, up to 500 ml of the liquid to be aerosolized accumulates in the lungs. In some embodiments, up to one liter of the liquid to be aerosolized accumulates in the lungs. In some embodiments, up to two liters of the liquid to be aerosolized accumulates in the lungs. In some embodiments, energy including ultrasound, mechanical thumping or other mechanical energy is applied to the outside of the body in the chest region to assist in dislodging lung contaminants. In some embodiments, energy including any ultrasound, acoustic energy, or mechanical energy is applied inside of the pulmonary tissue to assist in dislodging lung contaminants. In some embodiments, a suction tube is inserted into the pulmonary tissue to remove the fluid and the contaminants.

Disclosed herein is a method of treating a subject in need thereof, comprising administering a composition of any preceded claim can include but not limited to administer or by a meter dose inhaler. In some embodiments, the method comprises administering a dose of 1-2 mL, 2-3 mL, 3-4 mL, 4-5 mL, 5-6 mL, 6-7 mL, 7-8 mL, 8-9 mL, 9-10 mL at least once or twice or thrice daily. In some embodiments, the method can be administered upon the onset of the hypoxia symptom when SpO2 level falls below 95%, 94%, 93%, 92%, 90% or 80%. In some embodiments, the method can be administered to a subject who has chronic asthma or COPD and exposed to a community spread pandemic or epidemic.

### REFERENCES

1. Lehmler, H. Anti-inflammatory effects of fluorocarbon compounds. Expert Rev. of Respir. Med. 2, 273-89 (2006).
2. Murgia, X., et al. Aerosolized fluorocarbon improves gas exchange and pulmonary mechanics in preterm lambs with severe respiratory distress syndrome. Pediatr. Res. 72, 393-399 (2012).
3. Wang, X. et al. Sustained improvement of gas exchange and lung mechanics by vaporized fluorocarbon inhalation in piglet acute lung injury model. Clin. Respir. J. 8, 160-166 (2014).
4. Kacmarek, R. , et al. Partial Liquid Ventilation in Adult Patients with Acute Respiratory Distress Syndrome. Am. J. Respir. Crit. Care Med. 173, 882-889 (2005).
5. Varon, J., et al. Therapeutic Hypothermia; past, present and future. Chest. 133, 1267-1274 (2008).

1. A system for delivering aerosolized fluorocarbons, the system comprising:
a container;
an aerosolizer fluidically coupled to the container and configured to aerosolize fluids from the container; and
an introduction assembly fluidically coupled to the aerosolizer and configured to introduce aerosolized fluids into the pulmonary tissue of a subject.

2. The system of claim 1, wherein the container is a first container, and the system further comprises a second container and a mixer, the mixer fluidically coupled to the first container and to the second container, and the aerosolizer fluidically coupled to the mixer, the first container, and the second container.

3. The system of claim lcither of claim 1, further comprising a collection system and a return system, the collection system fluidically coupled to the introduction assembly and configured to collect and condense exhalate or expired fluid from the pulmonary tissue of the subject into a condensate, the return system fluidically coupled to the collection system and configured to receive the condensate from the collection system and return the condensate to the aerosolizer.

4. The system of claim 3, wherein the collection system further comprises a condenser.

5. The system of claim 3, or wherein the collection system further comprises a collection tube fluidically coupled to the introduction assembly and the collection system, and a one-way valve or flow diverter fluidically coupled to the introduction assembly but configured to direct exhalate or expired fluid from the pulmonary tissue of the subject into the collection tube.

6. The system of claim 3, further comprising a flow diverter fluidically coupled to the introduction assembly and the collection system, the flow diverter configured to pass aerosolized fluids from the aerosolizer into the introduction assembly, the flow diverter further configured to pass exhalate or expired fluid from the pulmonary tissue to the collection system.

7. The system of claim 3, wherein the return system comprises a separation system fluidically coupled to the collection system, the separation system configured to remove water, exhaled gasses, and contaminants from the condensate before delivering the condensate to the return system.

8. The system of claim 3, wherein the return system comprises a filter for removing water, contaminants, or both from the condensate.

9. The system of claim 1, wherein the container is a first container, and the system further comprises a second container, wherein the first container comprises a first fluorocarbon (FC) and the second container comprises a second FC.

10. The system of claim 9, wherein the first FC comprises a boiling point below 37° C.

11. The system of claim 9, wherein the first container and the second container are housed one or more disposable cartridges.

12. The system of claim 1, wherein the aerosolizer is configured to aerosolize FCs at a rate of at least 0.5 mL/minute.

13. The system of claim 12, wherein the aerosolizer is configured to aerosolize FCs at a rate of at least 2 mL/min.

14. The system of claim 1, further comprising a gas delivery regulator fluidically coupled to the aerosolizer and a gas cannister, the gas delivery regulator configured to control the flow of a delivery gas to the aerosolizer.

15. The system of claim 1, wherein the introduction assembly comprises a mask.

16. The system of claim 15, wherein the introduction assembly further comprises at least one one-way valve or flow diverter.

17. The system of claim 1, wherein the introduction assembly comprises a ventilator, and the ventilator comprises an exhalation pressure control system.

18. The system of claim 17, further comprising a collection system fluidically coupled to the ventilator, the collection system comprising a condenser configured to condense and collect exhalate or expired fluid from the pulmonary tissue of the subject into a condensate, wherein the condenser is configured not to interfere with the exhalation pressure control system of the ventilator.

19. A method of delivering aerosolized fluorocarbon (FC) to the pulmonary tissue of a subject, the method comprising:
aerosolizing a FC using an aerosolizer;
delivering the FC to the pulmonary tissue of the subject; and
contacting the pulmonary tissue of the subject with the FC.

20. The method of claim 19, wherein the FC is a first FC, and aerosolizing the first FC further comprises mixing the first FC with a second FC and aerosolizing a mixture of the first FC and the second FC, wherein the first FC is selected to have a first boiling point and the second FC is selected to have a second boiling point, and further comprising balancing the ratio of the first FC to the second FC to engineer a desired boiling point, enthalpy of vaporization, degree of cooling, degree of warming, cooling rate, or warming rate upon contact of the mixture with the pulmonary tissue.

21. The method of claim 19, further comprising collecting exhaled or expired fluid from the pulmonary tissue of the subject, condensing the exhaled or expired fluid via a condenser, and returning condensed FC to the aerosolizer to be recycled back to the pulmonary tissue of the subject.

22. The method of claim 21, further comprising separating the first FC from one or more of water, contaminants, and exhaled gas in the exhaled or expired fluid before returning condensed FC to the aerosolizer.

23. The method of claim 21, wherein condensing the exhaled or expired fluid does not change an exhalation pressure control on a ventilator to which the condenser is coupled.

24. The method of claim 19, further comprising mixing the FC with a delivery gas, wherein mixing the FC and the delivery gas occurs before aerosolizing the FC.

25. The method of claim 19, further comprising mixing the FC with a delivery gas, wherein mixing the FC and the delivery gas occurs after aerosolizing the FC.

26. The method of claim 19, wherein aerosolizing the FC comprises aerosolizing at a rate of at least 0.5 mL/minute.

27. The method of claim 19, wherein aerosolizing the FC comprises aerosolizing at a rate of at least 2 mL/minute.

28. The method of claim 19, further comprising removing residual FC from the pulmonary tissue.

29. The method of claim 19, wherein contacting the pulmonary tissue further comprises cooling the pulmonary tissue.

30. The method of claim 29, wherein the FC has a boiling point below 37° C.

31. The method of cithcr claim 29 or claim 30, wherein the subject is undergoing surgery, has an injury, and/or suffers from ARDS, stroke, heart attack, traumatic brain injury, acute encephalitis, neonatal hypoxia, and/or near drowning, and cooling the pulmonary tissue provides a therapeutic benefit for the subject.

32. The method of claim 29, wherein the pulmonary tissue is cooled by a range of from 2° C. to 6° C.

33. The method of claim 32, further comprising reducing inflammation of the pulmonary tissue.

34. The method of claim 29, further comprising preserving the pulmonary tissue of a deceased subject for transplantation.

35. The method of claim 34, wherein the pulmonary tissue is cooled by a range of from 17° C. to 33° C.

36. The method of claim 29, wherein cooling the pulmonary tissue comprises cooling at a rate of from 0.05° C./minute to 3° C./minute.

37. The method of claim 29, wherein cooling the pulmonary tissue comprises delivering the FC at a rate of at least 2 mL/minute.

38. The method of claim 19, further comprising performing a lung lavage by delivering mechanical energy to the pulmonary tissue to dislodge one or more of mucus, pus, pollutants, foreign materials, or debris.

39. The method of claim 38, wherein the FC has a boiling point above 37° C.

40. The method of claim 38, wherein delivering the FC to the pulmonary tissue of the subject comprises delivering up to 500 mL of aerosolized FC.

41. The method of claim 19, wherein delivering the FC to the pulmonary tissue of the subject further comprises mixing the FC with an active pharmaceutical ingredient.

42. A method of preserving cadaver pulmonary tissue, the method comprising:
selecting a FC with a boiling point below 37° C.;
aerosolizing the FC using an aerosolizer;
delivering the FC to the pulmonary tissue of the subject;
contacting the pulmonary tissue of the subject with the FC; and
cooling the pulmonary tissue;
wherein the pulmonary tissue is cooled to a range of from 4° C. to 20° C. to preserve it for transplantation.

43. The method of claim 42, wherein cooling the pulmonary tissue comprises cooling at a rate of from 0.5° C./minute to 3° C./minute.

44. The method of claim 42, wherein cooling the pulmonary tissue comprises delivering the FC at a rate of at least 2 mL/minute.
